SEQUENCE LISTING (33 sequences)

SEQ ID NO: 1 - protein, 9 aa, Homo sapiens
AGQSLLKDE

SEQ ID NO: 2 - protein, 9 aa, Homo sapiens
AWPFTCLPL

SEQ ID NO: 3 - protein, 9 aa, Homo sapiens
WPFTCLPLG

SEQ ID NO: 4 - protein, 9 aa, Homo sapiens
SGNRASLYS

SEQ ID NO: 5 - protein, 9 aa, Homo sapiens
LYSFPEPEA

SEQ ID NO: 6 - protein, 9 aa, Homo sapiens
FPEPEAAQP

SEQ ID NO: 7 - protein, 9 aa, Homo sapiens
VEVLVDLFL

SEQ ID NO: 8 - protein, 9 aa, Homo sapiens
EKVKRKKNV

SEQ ID NO: 9 - protein, 9 aa, Homo sapiens
KVKRKKNVL

SEQ ID NO: 10 - protein, 9 aa, Homo sapiens
RKKNVLRLC

SEQ ID NO: 11 - protein, 9 aa, Homo sapiens
NLTHVLYPV

SEQ ID NO: 12 - skipped

SEQ ID NO: 13 - protein, 15 aa, Homo sapiens
TSPRRLVELAGQSLL

SEQ ID NO: 14 - protein, 15 aa, Homo sapiens
RLVELAGQSLLKDEA

SEQ ID NO: 15 - protein, 15 aa, Homo sapiens
PFTCLPLGVLMKGQH

SEQ ID NO: 16 - protein, 15 aa, Homo sapiens
LPLGVLMKGQHLHLE

SEQ ID NO: 17 - protein, 15 aa, Homo sapiens
VLMKGQHLHLETFKA

SEQ ID NO: 18 - protein, 15 aa, Homo sapiens
DVLLAQEVRPRRWKL

SEQ ID NO: 19 - protein, 15 aa, Homo sapiens
DELFSYLIEKVKRKK

SEQ ID NO: 20 - protein, 15 aa, Homo sapiens
SYLIEKVKRKKNVLR

SEQ ID NO: 21 - protein, 15 aa, Homo sapiens
EKVKRKKNVLRLCCK

SEQ ID NO: 22 - protein, 15 aa, Homo sapiens
CCKKLKIFAMPMQDI

SEQ ID NO: 23 - protein, 15 aa, Homo sapiens
AMPMQDIKMILKMVQ

SEQ ID NO: 24 - protein, 15 aa, Homo sapiens
QDIKMILKMVQLDSI

SEQ ID NO: 25 - protein, 14 aa, Homo sapiens
SPLGQMINLRRLLL

SEQ ID NO: 26 - protein, 15 aa, Homo sapiens
EEQYIAQFTSQFLSL

SEQ ID NO: 27 - protein, 15 aa, Homo sapiens
LERLAYLHARLRELL

SEQ ID NO: 28 - protein, 509 aa, Homo sapiens
  MISC_FEATURE (1)..(609): Accession: P78395.1 GI: 6685631
MERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 29 - protein, 15 aa, Homo sapiens
SPEKEEQYIAQFTSQ

SEQ ID NO: 30 - protein, 15 aa, Homo sapiens
ASLYSFPEPEAAQPM

SEQ ID NO: 31 - protein, 15 aa, Homo sapiens
SFPEPEAAQPMTKKR

SEQ ID NO: 32 - protein, 15 aa, Homo sapiens
EQPFIPVEVLVDLFL

SEQ ID NO: 33 - protein, 15 aa, Homo sapiens
IPVEVLVDLFLKEGA

(19) United States
(12) Patent Application Publication
(10) Pub. No.: US 2023/0190902 A1
(43) Pub. Date: Jun. 22, 2023

(54) IDENTIFICATION OF HLA-RESTRICTED PRAME PEPTIDE EPITOPES, PRAME-SPECIFIC T CELLS SUITABLE FOR "OFF-THE-SHELF" TREATMENT OF CANCER EXPRESSING PRAME

(71) Applicant: Children's National Medical Center, Washington, DC (US)

(72) Inventors: Catherine Mary Bollard, Washington, DC (US); Patrick Hanley, Washington, DC (US); Conrad Russell Y. Cruz, Washington, DC (US); Maja Stanojevic, Washington, DC (US); Amy Hont, Washington, DC (US)

(21) Appl. No.: 17/926,188

(22) PCT Filed: May 28, 2021
(86) PCT No.: PCT/US2021/035008

§ 371 Date: Nov. 18, 2022

Related U.S. Application Data
(60) Provisional application No. 63/031,929, filed on May 29, 2020.

(51) Int. Cl. A61K 39/00; A61K 39/39; A61P 35/00

(52) U.S. Cl. CPC A61K 39/001189; A61K 39/39; A61K 39/001186; A61K 39/001188; A61K 39/001182; A61K 39/00116; A61K 39/001151; A61K 39/001194; A61K 39/001152; A61K 39/001192; A61K 39/001184; A61K 39/001164; A61P 35/00; A61K 2039/5158; A61K 2039/55561; A61K 2039/55516; A61K 2039/55533; A61K 2039/55538; A61K 2039/55527

(57) ABSTRACT

The invention pertains to a method for treating a cancer which expresses PRAME using T cells which recognize specific peptide epitopes of PRAME, to a method for producing T cells that target cancer cells expressing PRAME, the peptide epitopes of PRAME themselves and to compositions and methods of treatment using these peptides.

### CROSS-REFERENCE TO RELATED APPLICATIONS

This application claims priority to U.S Provisional 63/031,929, filed May 29, 2020 which is incorporated by reference for all purposes.

### REFERENCE TO A SEQUENCE LISTING

In accordance with 37 CFR 0.52(0(5), the present specification makes reference to a Sequence Listing (submitted electronically as a .txt file named “530688WO_ST25.txt”. The .txt file was generated on May 14, 2021 and is 10.366 bytes in size. The sequence listing forms are integral part of this description/disclosure and the entire contents of the Sequence Listing are herein incorporated by reference.

### BACKGROUND OF THE INVENTION

Field of the Invention. The present disclosure relates to the field of medicine and immunology. In particular it relates to peptide epitopes derived from PRAME (PReferentially expressed Antigen in MElanoma cells) (“PRAME”) antigen that are restrictable on HLA class 1 or HLA class 2 molecules, to T cells -ecognizing these restricted PRAME. peptide epitopes, to immunogens or vaccines comprising these peptide epitopes, and to methods for preventing or treating neoplasms or cancers which express PRAME using PRAME-specific T cells or using these PRAME derived peptide epitopes.

Description of Related Art. The tumor-associated antigen PRAME was originally, identified as an antigenrecognized by cytotoxic T lymphocytes capable of lysing melanoma cells, Ikeda et al., 1997; 6:199-208) The tumor antigen PRAME is now known to be overexpressed in a wide variety of human cancers including lymphoid and myeloid malignancies and solid tumors. Overexpression of PRAME, as is frequently observed in human malignancies, is considered to provide tumor cells growth and su al advantages by antagonizing retinoic acid receptor (RAR) signaling. Over-expression may break immunological tolerance to PRAME which is expressed in some humantissues such as testicular tissue.

Despite many publications that indicate the potential of PRS ME as a tumor antigen and attractive candidate target of eliciting anti-tumor cell immune responses and preparing anti-tumor vaccines, little data are available that identify and show the inummogenicity of PRAME derived peptide epitopes, which is needed to establish an effective anti-tumor T-cell response.

Prior studies have shown that some autologous T cells can stabilize or maintain durable remissions of some types of cancers. Howe er, the production of sufficient, clinically relevant numbers of autologous cells that recognize cancer antigens is problematic, especially for patients undergoing pharmacological or radiation therapies who are lymphopenic or immunosuppressed.

The present disclosure addresses these problems by identifying PRAME-derived peptide epitopes that when presented by HLA class 1 or HLA class 2 molecules are recognized by T cells that target cancer cells expressing PRAME. It also provides a method for producing clinically relevant populations of cells that target cancer cells expressing PRAME by priming and expanding precursor T cells or T cells, such as those obtained from PBMCs of healthy donors. These T cell populations, which target cancers that express PRAME, may be viably stored or cryogenically frozen and rapidly deployed “off-the-shelf”for treatment of cancers demanding immediate treatment including relapsing cancers e. pressing PRAME.

### BRIEF SUMMARY OF THE INVENTION

One aspect of the invention is directed to a. population of T cells which target cancer cells expressing PRAME via one or more of the peptide epitopes of PRAME disclosed here.

A related aspect is directed to a method for treating or preventing a cancer that expresses PRAME by adoptively transferring or infusing a population of T cells recognizing one or more of the PRAME epitopes disclosed here.

Another aspect of the disclosure is directed to the peptide epitopes of PRAME which have been identified by the inventors, such as those described by SEQ ID NOS: 1-26 or their modified forms, and to compositions containing them.

Another aspect of the disclosure is directed to a. method for making, by priming and expanding, or by expanding, T cells that recognize the peptide epitopes of PRAME described herein.

A related aspect of the disclosure is directed to a T cell bank which viably stores T cells recognizing the peptide epitopes disclosed herein for rapid off-the-shelf-use in treating patients having cancers expressing PRAME.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings below.

FIG. 1A. Total cell number of PRAME-specific T-cells generated from 12 healthy donors (Product numbers 1-12) on days 0, 7, 14 and 21. PBMCs were primed with PRAME-pulsed dendritic cells (“DCs”) on day 0 and restimulated on days 7 and 14.

FIG. 1B, Phenotyping analysis of PRAME-specific T-cell products, assessed by flow cytometry showing a mixture of both CD4⁺ and CDS8⁺ T-cells with a balanced predominance of central memory (CD3⁺;CD45RO⁺CD62L⁺) and effector memory (CD3⁺CD45RO⁺CD62L⁻) phenotype. No outgrowth of NK. (CD3⁺CD∵RO⁺CD56⁺) or NKT (CD3⁺CD16⁺CD56⁺) cells was observed.

FIG. 1C shows specificity of the T-cell products for control antigen (actin) vs. the PRAME antigen as measured by the IFN-γELISpot assay.

FIGS. 2A-1 to 2A-3 (panel) compare Wilms tumor cell lines 17,94 (FIG. 2A-1) with Wit49, which was derived from a primary lung metastasis of an aggressive Wilms tumor 2, (FIG. 2A-2) stained positive for PRAME by immunofluorescence, compared to pancreatic cells, negative control (FIG. 2A-3). This comparison shows that off-the-shelf tumor associated antigen T cells (“TAA T”) are cytolytic against Wilms tumor cell lines which express PRAME.

FIG. 2B-1 shows that TAA-T products are polyclonal and that they secrete cytokines upon stimulation with PRAME as measured by flow cytometry and intracellular cytokine staining.

FIG. 2B-2. Phenotyping analysis of PRAME-specific T-cell lines as assessed by flow cytometry showing a mixture of both CD4+and CD8+T-cells with a balanced predominance of central memory (CD3⁺CD45RO⁺CD62L⁺) and effector memory (CD3⁺CD45RO⁺CD62L⁻) phenotype. There was no outgrowth of NK (CD3⁻CD16⁺ CD56⁺), NKT (CD3⁺CD16⁺CD56⁺) and Treg (CD3⁺CD4^{+CD}25⁺CD127dim)

FIG. 2B-3 shows PRAME specificity by product. Specificity of the T-cell product for PRAME antigen was measured by IFN-γ ELISpot assay. Background levels of unstimulated T-cells and T-cells stimulated with actin (irrelevant antigen) were subtracted from the final results.

FIG. 2C shows that TAA-T products demonstrate specificity for PRAME as measured by IFN-γ ELISpot assay. Background response to actin was subtracted from the final results.

FIGS. 2D-2F describe PRAME-specific TAA-T products which demonstrate antigen-specific cytotoxicity to Wilms tumor cell line 17.94.

FIGS. 2G and 2H describe PRAME-specific TAA-T products which demonstrate antigen-specific cytotoxicity to Wit49 cell line.

FIG. 2I shows the absent/non-specific activity of TAA-T cells to autologous PHA (phytohemagglutinin) blasts or PBMCs.

FIG. 3A. IFN-γ production by T-cells in response to PRAME peptide pool stimulation.

FIG. 3B. T-cell responses to single 15-mer PRAME peptides, present in the mini-pools, which were evaluated by IFN-γ ELISpot assay,

FIG.3C shows that single peptides 18, 19, 35, 36, 37, 43, 44, 48, 49, 51, 124, and 125 were immunogenic.

FIG. 3D (chart) describes HLA-I-restricted epitopes 36 (SEQ ID NO: 30), 37 (SEQ ID NO: 31), 43 (SEQ ID NO: 32), 44 (SEQ ID NO: 33 that were determined by measuring IFN-γ and TNE-α, release by CD8⁺ T-cells.

FIG. 3E (chart) shows that CD4⁺ T-cells showed no specificity for HLAA-restricted epitopes 36 (SEQ ID NO: 30), 37 (SEQ ID NO: 31), 43 (SEQ ID NO: 32), 44 (SEQ ID NO: 33).

FIG. 3F describes the minimal 9-mer peptides-VEVLVDLFL (SEQ ID NO: 7) and FPEPEAAQP (SEQ ID NO: 6)-that were determined by IFN-γ ELISpot assay. As shown left to right: VEVLVDLFL, (SEQ ID NO: 7), PVEVLVDLF (SEQ ID NO: 33, residues 2-10), IPVEVLVDL (SEQ ID -NO: 33, residues 1-9), PEPEAAQP (SEQ ID NO: 30, residues 7-14), FPEPEAAQP (SEQ ID NO: 6), and SFPEPEAAQ (SEQ ID NO: 30, residues 1-9).

FIG. 3G confirms HLA restriction of FPEPEAAQPM (SEQ ID NO: 6) using anti-HLA-B*35 antibody.

FIG. 4A describes ITN-γ production by T-cells in response to PRAME peptide pool stimulation. As shown, the T cell product recognized four mini pools 2, 11, 17 and 18.

FIG. 4B describes T-cell responses to single PR IF peptides, present in the mini-pools 2, 11, 17, and 18 which were evaluated by the liFiN-7 ELISpot assay.

FIG. 4C demonstrated recognition of single peptides 50 EKVKRKKNVLRLCCK (SEQ ID NO: 21), 70 SPEKEEQYIAQFTSQ (SEQ ID NO: 29) and 71 EEQYIAQFTSQFLSL (SEQ ID NO: 26).

FIG. 4D (chart) confirms HLA class 1 restriction of peptides 50 and 71 as determined by release of IFN-γ and TNF-α, by CD4⁺ T cells. As shown: Peptide 50 EKVKRKKNVLRLCCK (SEQ ID NO: 21), 70 SPEKEEQYIAQFTSQ (SEQ ID NO: 29) and 71 EEQYIAQFTSQFLSL (SEQ ID NO: 26).

FIG. 4E (chart). CD8⁺ T cells showed no specificity to peptides 50 and 71 as determined by absence of release of IFN-γ and TNF-α, in response to class II -restricted peptide epitopes. As shown: Peptide 50 EKVKRKKNVLRLCCK (SEQ ID NO: 21), 70 SPEKEEQYIAQ-FTSQ (SEQ ID NO: 29) and 71 EEQYIAQFTSQFILSL (SEQ ID NO: 26),

FIG. 5 (chart) shows cytotoxic (CD8⁺) and helper (CD4⁺) T-cell responses to PRAME, epitope RLVELAGQSLLKDEA (SEQ ID NO: 14) Results showed that this peptide activated both CD8⁺ and CD4⁺ T-cells.

FIG. 6 depicts the locations of the identified CD4 and CD8-restricted epitopes within PRAME protein.

FIG. 7A depicts peptide libraries of 15-mer peptides overlapping by 11 amino acids spanning the entire sequence of the PRAME protein consisting of 509 amino acids. The sequence of the first two 15-mors with 11 amino acid overlap is illustrated as one example.

FIG. 7B describes 23 peptide pools comprising 10-12 peptides that were prepared so that each 15-mer peptide was included in only two pools.

FIG. 8 describes subpopulations of T cells and the markers they express.

### DETAILED DESCRIPTION OF THE INVENTION

The inventors sought to identify new PRAME epitopes thus extending the repertoire of HLA-restricted PRAME peptide epitopes beyond the few already characterized. While autologous T cell products demonstrate an excellent safety profile, patients are often subjected to salvage therapies over the 4 to 8 weeks of waiting period for T cell product generation. This is quite detrimental when urgent cancer therapy is required, for example, to treat a cancer in its early stages, in early relapse, or prior to metastasis.

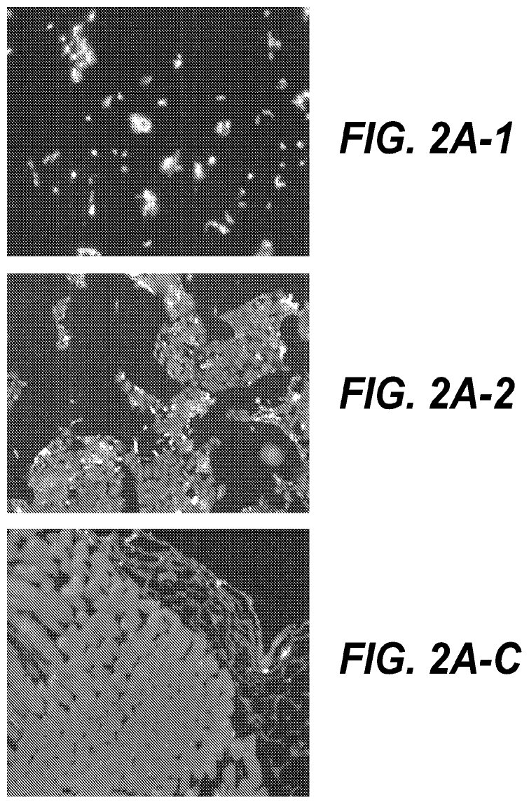

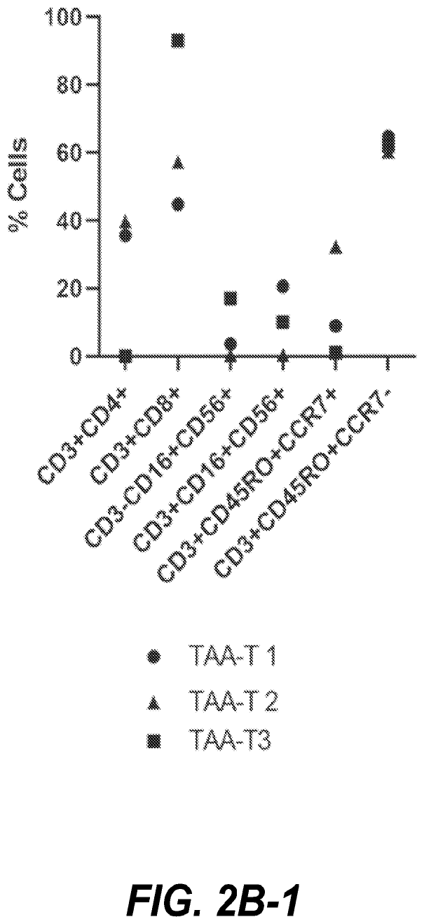

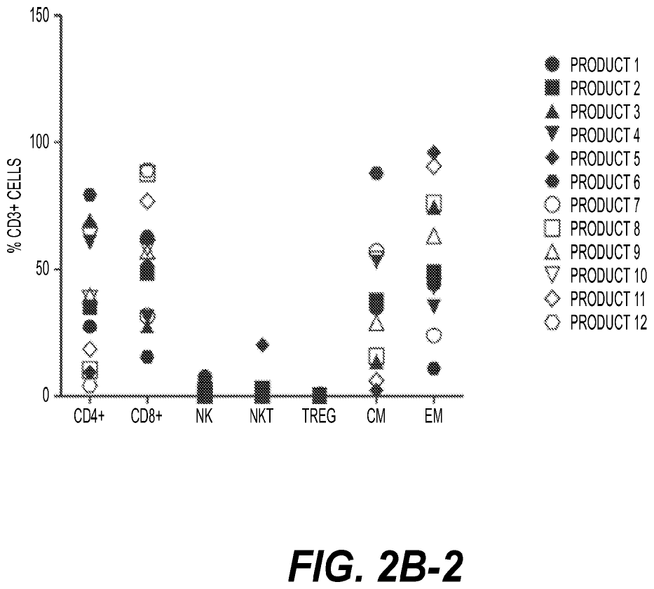

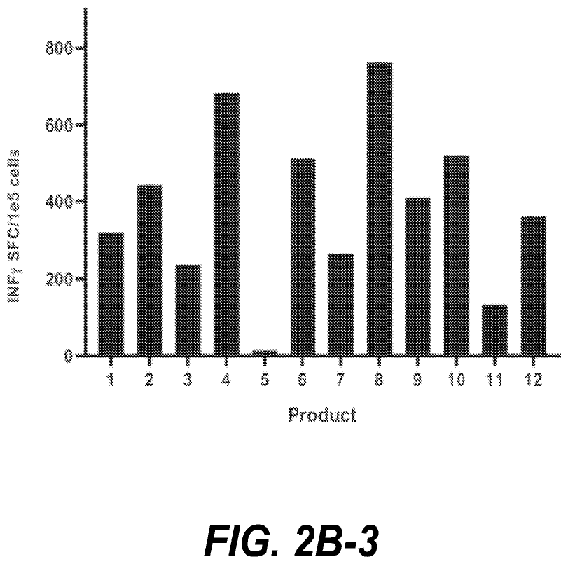

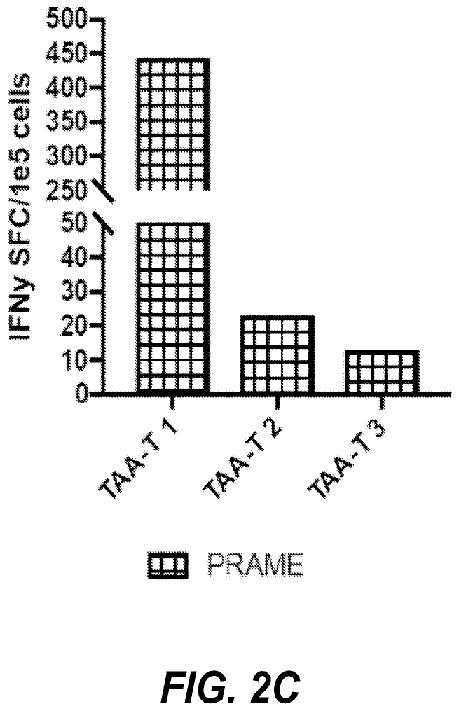

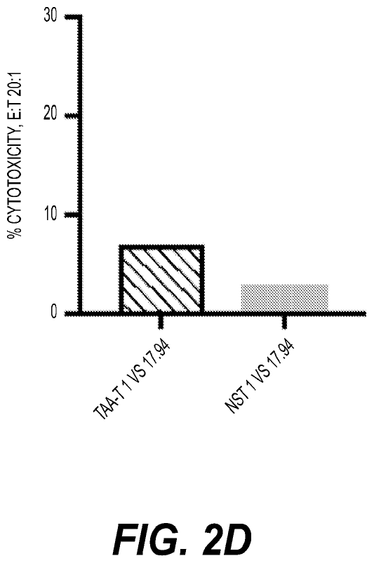

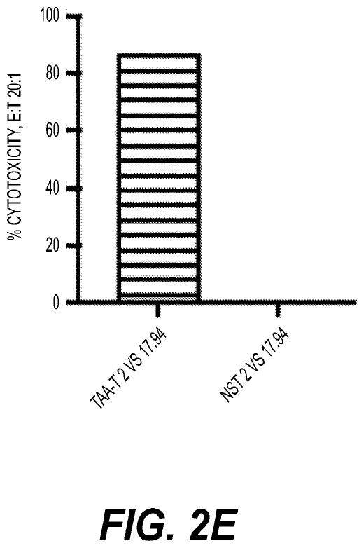

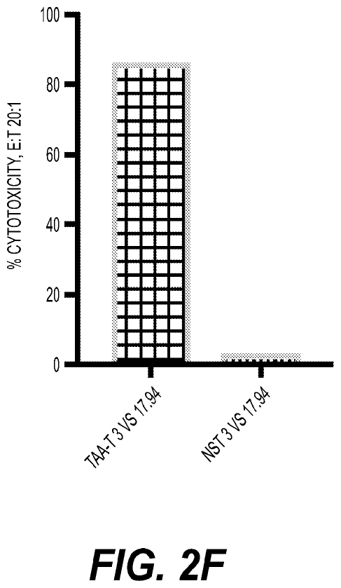

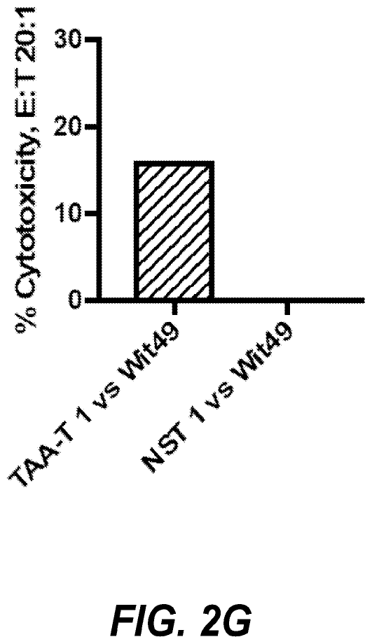

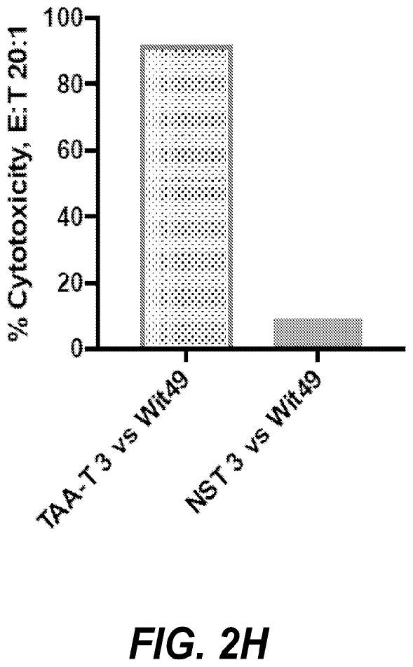

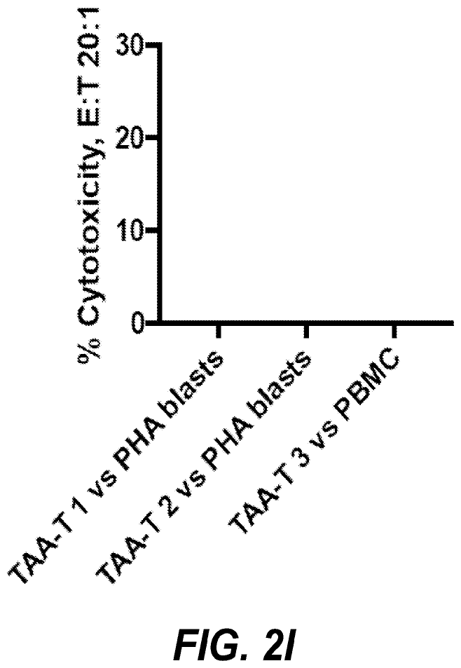

The inventors have now shown that PRAME-specific T-cells generated from healthy donors demonstrate tumor-specific cytotoxicity in vitro to partially HLA-matched tumor cell lines; see FIGS. 2A1-2C. The tumor-specific cytotoxicity demonstrated here permits the PRAME-specific T cells recognizing epitopes disclosed herein to be used to treat cancer patients with an off-the-shelf, partially HLA-matched allogeneic product for rapid “on demand”treatment.

The development of third party PRAME-specific T-cell therapeutics for the treatment of Wilms tumor and other PRAME-expressing cancers would therefore overcome the inherent and practical limitations of using autologous patient-derived products.

The importance of PRAME epitope identification extends beyond the manufacture of a robust third party T-cell bank. For example, T-cells specific for identified epitopes can be tracked using multimers both in vitro and in vivo, and even in situ as described by and incorporated by reference to Abdelaai, IL M. et at, Detection of Antigen-Specific T Cells Using In Situ MHC Tetramer Staining, 2019, 20(20): 5165, [421].

Moreover, identification of the TCRs responding to these PRAME specific epitopes as described by and incorporated by reference to Shao, H.W., et al., CANCER. LETTS., 2015, 363(1).83-91, allows the use of TCR sequencing to track unique T-cell clones that may contribute to the anti-tumor response, and the construction of an engineered αβTCR for gene-modified T-cell therapies targeting specific individual TAA epitopes

Finally, identifying the breadth of the epitope specific T-cell response is also a critical step for effective vaccine design targeting PRAME using methods described by and incorporated by reference to Oka, Y. et al., ONCOL. RES. TREAT, 2017, 40:682-90.

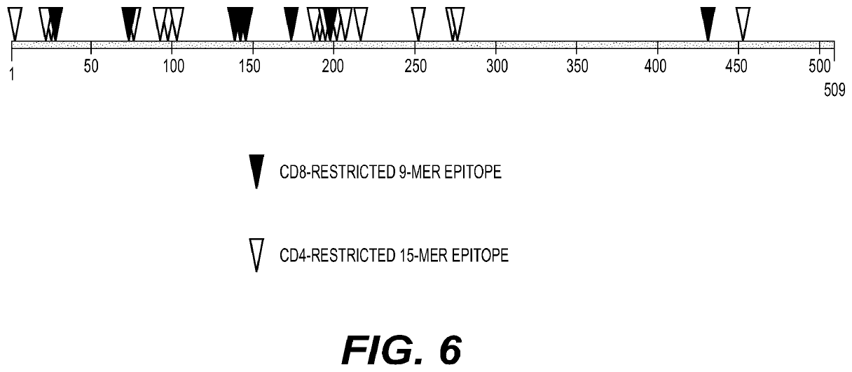

The inventors describe herein a series of novel MHC (MLA) class I and II epitopes specific for PRAME and use their ex vivo expansion protocol, which is incorporated by reference to Weber, G., et al., LEUKEMIA, 2013, 27(7);1538-47, to produce PRAME-specific cells to these epitopes. Class I epitopes from the 15-mer pools were confirmed using specifically manufactured 9-mer peptides and identified epitopes which spanned the entire sequence of PRAME protein as shown by FIG. 6, Allogenic healthy donor-derived T cells killed partially HLA matched tumor cells unlike T cells that did not specifically recognize PRAINTE. These results are consistent with the presence of elicited memory and effector memory T cell responses exhibiting activity against PRAMF-expressing targets.

Surprisingly, PRAME epitopes that simultaneously elicited both CD4⁺ and CD8⁺ T-cell responses were also identified. In other systems, such epitopes have been shown to be important for an effective cytotoxic T-cell response and persistence of adoptively transferred T-cells in vivo; see Castellino, F, et al., RN. Cooperation between CD4(+) and CD8(+) T cells: when, where, and how. ANNUAL REVIEW OF IMMUNOLOGY 2006;24:519-40 and Schoenberger SP, et al. I-cell help for cytotoxic T lymphocytes is mediated by CD40-CD40L interactions. Nature 1998;393(6684):480-3. Such epitopes may be used to prime or expand T cells that recognize PRAMF, enhance anti-PRAME cytotoxic responses, and provide long term anti-PRAME responses when adoptively transferred to a patient.

The inventors demonstrate herein how a third party tumor antigen-specific T-cell product targeting PRAME can be applied to the solid tumor setting. The identification of novel class I and class II HLA-restricted PRAME-specific T-cell epitopes which are represented in populations from different geographic regions (see allele frequencies.net) permits one to pick the most advantageous I-cell donor product for any given patient by ensuring that epitope specific responses are restricted to HLA allele or alleles shared between donor and recipient. For example, the AWPFTCLPL (SEQ ID NO: 2) peptide epitope is predicted to be an HLA-A*24-restricted I-cell epitope and the gene frequency of HLA-A*24 (A*24:02) is high in Asian and Hispanic populations suggesting selective use of this epitope in treating patients in those populations. Similarly, the peptide KVKRKICNVL (SEQ ID NO: 9) which is an HLA-B*08 (B*08:01)-restricted T-cell epitope. The HLA-B*08:01 allele is lhighly prevalent in individuals of Caucasian ancestry and common in Asian/Pacific :Islander and African American populations suggesting selective use of this epitope in treating patients in those populations.

Creating off-the-shelf products has many potential advantages since such products are readily available for the treatment of patients with aggressive disease o and readily available for patients where an autologous product cannot be manufactured.

Additionally, T-cell products derived from healthy donors are generally more reliably expanded to specific quality and potency specifications to facilitate the manufacture of large quantities of tumor-specific T-cells for the treatment of a broad number of solid tumors that express cancer testis antigens such as PRAME.

Embodiments of the invention include, but are not limited to, the following.

A method for eliciting an immune response in a subject having cancer expressing “PReferentially expressed Antigen in MElanoma cells”(“PRAME”) comptising administering T cells which recognize an epitope of PRAM present in a peptide having an amino acid sequence consisting of SEQ ID NO: 1, 2, 3, 4, 5, 6, 7, 8, 9, 10, or 11, or SEQ ID NO: 12, 14, 15, 17, 18, 19 or 20. Typically, the immune response elicited reduces the severity of the cancer, such as reducing its mass, growth rate, or rate of progression.

In one embodiment, the PRAMS-specific T cells are administered parenterally, for example, by intravenous infusion, intraperitoneal infusion, or other parenteral mode. T cells may also be infused or administered to a site of cancer.

In one embodiment of this method, the T cells are derived from a healthy donor who shares at least one HLA class 1 or HLA class 2 allele or HLA antigen with a patient or recipient. Alternatively, the T cells may be autologously derived from the patient or from frozen or stored bone marrow or cord blood of a patient. In a preferred embodiment, the T cells are obtained from a healthy donor who shares at least one HLA class 1 or HLA class 2 antigen with the subject.

In some embodiments, the T cells may be obtained from a donor who has had, or has, a cancer, such as a cancer expressing PRAME., and who shares at least one HLA class 1 or class 2 haplotype with the recipient or who coexpresses a FELA protein or antigen with the recipient.

In some embodiments, the T cells are obtained from peripheral blood mononuclear cells (PBMCs) or from tumor infiltrating lymphocytes of a donor or subject.

In some embodiments, the T cells are primed and expanded, or expanded, ex vivo or in vitro after recovering them from a donor. A donor may be the patient or a third party donor, often a genetically close family member.

In some embodiments, the T cells are produced by contacting naive T cells or T cell precursors, or alternatively, T cells that already recognize PRAME., with antigen presenting cells pulsed with at least one peptide of SEQ ID NOS: 1-27. In some embodiments, the T cells already recognize a peptide epitope of PRAME. such as the epitopes of SEQ ID NOS: 111, or more specifically, a peptide epitope of SEQ ID NO: 2, 6, 7, 8 or 9.

In some embodiments, the T cells recognize a peptide epitope of SEQ ID NO: 2 and said subject expresses HLA-A 24:02; or the T cells recognize a peptide epitope of SEQ ID NO:6 and said subject expresses HLA-B 35:03; or the T cells recognize a peptide epitope of SEQ ID NO: 9 and said subject expresses HLA-B 08:02.

In other embodiments, the T cells recognize a peptide epitope of PRAME in or on peptide consisting of SEQ ID NOS: 12 to 20, such as a peptide epitope of PRAME in a peptide consisting of SEQ ID NO: 12, 13, 19, 20, 22, 26, 27 or 28, or such as an epitope of PRAME in a peptide consisting of SEQ ID NO: 14.

In some embodiments, the T cells recognize a peptide epitope of SEQ ID NO: 13 and said subject expresses HLA-DRB1 01:01; the T cells recognize a peptide epitope of SEQ ID NO: 19 and said subject expresses HLA-DPA1 02:02; the T cells recognize a peptide epitope of SEQ ID NO: 19 and said subject expresses HLA-DP131 04:02; the T cells recognize a peptide epitope of SEQ ID NO: 20 and said subject expresses HLA-DRB1 11:03; or the T cells recognize a peptide epitope of SEQ ID NO: 20 and said subject expresses HLA-DRB1 15:02.

In some embodiments, the T cells comprise different T cell populations which each recognize a different epitope of PRAME. For example, the T cells may comprise populations that recognize two, three, four or more HLA class 1 restricted epitopes, T cells may comprise populations that recognize two, three, four or more HLA class 2 restricted epitopes, or comprise mixed populations of T cells which recognize a two, three, four or more class 1 and/or class 2 restricted epitopes, such as those of SEQ ID NOS: 1-26.

In some embodiments of this method, the T cells are Obtained from a T cell bank and are selected to comprise at least one, two , three, four, five; six, seven, eight or more HLA class 1 or HLA class 2 antigens shared by a donor and by the subject,

In other embodiments of this method the T cells are administered in a form of a composition which may further comprise an adjuvant. The adjuvant may be selected from anti-CD40 antibody, imiquimod, resiquimod, GM-CSF, cyclophosphamide; sunitinib, bevacizumab, interferon-alpha, interferon-beta. CpG oligonucleotides and derivatives, poly-CLC) and derivatives, RNA, sildenafil, particulate formulations with poly(lactide co-glycolide) (PLG), virosomes, interleukin (IL)-1 IL-2, IL-4, IL-7, IL-12, IL-13, IL-15, IL-21, and IL-23.

In some embodiments, the method is used to treat at cancer that is a hetnatopoietic neoplasia.

In other embodiments, the method is used to treat or prevent relapse of acute myeloid leukemia, chronic myelogenous leukemia, chronic lymphocytic leukemia, relapsed leukemia, residual disease, such as residual disease after drug, irradiation, immunological or other types of cancer treatment.

In some embodiments, the method is used to treat cancer that is a solid cancer. In some embodiments, the method is used to treat skin cancers or melanoma. In other embodiments, the method is used to treat cancer selected from the group consisting of melanoma, lymphoma, papillomas, breast or cervical carcinomas, acute and chronic leukemia, medulloblastoma, non-small cell lung carcinoma, head and neck cancer, renal carcinoma, pancreatic carcinoma, prostate cancer, small cell lung cancer, multiple myeloma, sarcomas and hematological malignancies like chronic myeloid leukemia and acute myeloid leukemia.

In some embodiments, the subject is a patient who has been treated for cancer and has minimal residual disease, such as a patient undergoing chemotherapy, radiation therapy, or alternate immunotherapy.

In some embodiments, the method uses T cells that further comprise a population of T cells recognizing at least one antigen selected from the group consisting of NYESO, MAGE A4, MAGE A3, MAGE A1, Survivin, WT1, neuroela.stase, proteinase 3, p53, CEA, claudin6, Historic H1Histone H2, Histone H-3, Histone H4, MART₁, gp100, PSA, SOX2, SSX2, Nanog, Oct4, Myc, and Ras. In one embodiment, the method involves administering populations of T cells comprising, consisting essentially of, or consisting of those recognizing PRAME. in combination with T cells recognizing other tumor-associated antigens, including swvivin, MAGE-A3, MAGE-A4, other MAGE, NY-ESO-1 SSX-2, AML1ETO, DEK-CAN, PML-pRaR-alpha, Flf3-ITD and/or NPM1.

Another aspect of this technology is a method for producing T cells which recognize PRAME comprising, consisting essentially of, or consisti.ng of contacting a T cell or precursor T cell with an antigen presenting cell that presents at least one epitope of PRAME, and recovering a population of cells recognizing PRAME; wherein said epitope of PRAME is present in a peptide having an amino acid sequence consisting of one or more of SEQ ID NOS: 1-26 or other peptides disclosed herein.

In some embodiments of this method, the cell or precursor cells will be contacted with antigen presenting cells and PRAME peptides using the steps disclosed by U.S. Pat. Nos. 9,885,021, 10,934,525, or by PCT/US2016/023413 which steps may be modified to replace an overlapping peptide library with a less complex mixture of one, two, three or more peptide epitopes of PRAME such as those described by SEQ. ID NOS: 1-26.

In some embodiments, unmodified peptides comprising, consisting essentially of, or consisting of the amino acid sequences disclosed herein are used in this method; in other embodiments modified peptides, such as peptides having 1, 2 or 3 deletions, insertions or substations of amino acid residues in SEQ ID NOS: 1-26 or other peptides disclosed herein; or covalently modified peptides may be used in this method.

In some embodiments of this method, the T cell or precursor T cell and said antigen presenting cell are autologous. In other embodiments of this method the T cell or precursor T cell and said antigen presenting cell share at least on, two, three, four, five, six, seven, eight or more HLA class 1 or HLA class 2 antigens.

In some embodiments of this method the T cells or precursor T cells are naïve to PRAME. In other embodiments of this method, the T cells or precursor cells are memory T cells or effector T cells which recognize PRAME or other cells or T precursor cells not naïve to PRAMF or recognize PRAME epitopes when displayed by somatic or antigen presenting cells.

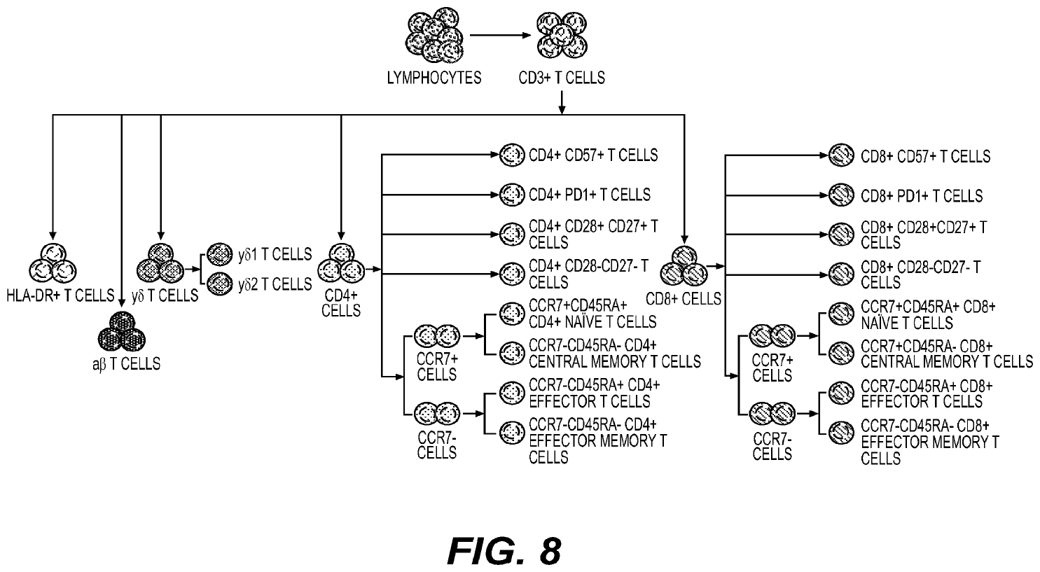

This method may further comprise separating the T cells which recognize by PRAME into subpopulations of T cells expressing one or more markers distinctive for that subpopulation, for example, as shown by FIG. 8 Separation may be performed using methods known in the art including cell sorting, flow cytometry, or isolation of subpopulations based on different density, or differential expression of cell markers. Preferably, adoptive transfer of a polyclonal population of CD4⁺ and CD8⁺ PRAME-specific cells is used in a third-party off-the-shelf setting, in which the T cells have epitope-specific activity through shared HLA allele(s) with the recipient, to support the in vivo persistence and expansion of transferred T cells. The ability to select PRAME-specific T cell products sharing HLA allele(s) with a patient, in a third party, off-the-shelf setting provides a clinical advantage over prior methods that do not provide ready access to these T cell products.

Some embodiments of this method further cotnprise suspending the T cells which recognize PRAME, in a storage buffer or in a cryogenic medium, and storing or freezing and thawing viable cells for later use. Cryogenic media for freezing and thawing T cells are known and commercially available for lxample from Thermofisher and methods for freezing and recovering viable T cells are known. Culture media and media components for growing T cells are also known and commercially available, for example, from Thermofisher (hypertext transfer protocol secure://www.thermofisher.com/us/en/home/life-science/cell-culture/mammalian-cell-culture/specialty-media/t-cell-media.html (last accessed May 11, 2021, incorporated by reference) or Cell Culture Dish, hypertext transfer protocol secure://cellculturedish.com/t-cell-media-comprehensive-guide-key-components/(last accessed May 11, 2021, incorporated by reference).

Another aspect of this disclosure comprises an isolated population of T cells that recognizes a peptide epitope of PRAMLE described by any one of the peptides of SEQ. ID NOS: 1-26 (or their modified forms) when presented by an HLA class 1 or HLA class 2 protein in combination with an artificial medium or carrier that maintains viability of the T cells. In some embodiments, the composition further comprises an adjuvant or a cytokine.

Another aspect of the disclosure is use of a population of T cells that recognize a peptide epitope of PRAME. described by any one of the peptides of SEQ ID NOS: 1-26 when presented by an HLA class 1 or HLA class 2 protein for preparation of a medicament to treat a cancer expressing PRAME.. This use may be directed to treatment of a cancer that expresses PRAME or is selected from the group consisting of melanoma, lymphoma, papillomas, breast or cervical carcinomas, acute and chronic leukemia, medulloblastoma, non-small cell lung carcinoma, head and neck cancer, renal carcinoma, pancreatic carcinoma, prostate cancer, small cell lung cancer, multiple myeloma, sarcomas and hematological malignancies like chronic myeloid leukemia and acute myeloid leukemia.

Another aspect of this technology is directed to a peptide or covalently modified peptide comprising or incorporating an amino acid sequence of any one of SEQ ID NOS: 1-26 or other peptides disclosed herein.

A peptide comprising SEQ ID NO: 1-26 may be covalently modified or engineered to improve its pharmacokinetic or pharmacodynamics properties, such as to increase its half-life in vivo or in viiro or resistance to excretion or degradation or its interaction with HLA n olecules and T cell receptors.

A modification may be a covalent modification of the peptide's N- or C-terminal, covalent conjugation to PEG, an adjuvant, or another carrier, or the incorporation of one or more D-amino acid residues into the sequence.

A peptide complex comprising a peptide, such as those of SEQ ID NOS: 1-26, may be formed by non-covalently binding a peptide to another moiety such as a carrier, adjuvant or substrate. In some embodiments a peptide is altered by non-covalently binding it to a carrier, adjuvant or substrate such as to PEG, BSA, or MAI, A peptide of SEQ ID NOS; 1-26 may form a non-covalent complex with an MHC class I or class II molecule or a complex with a cell membrane or cell comprising MHC class 1 or 2 molecules.

A modification may also involve deleting, substituting or inserting at least 1, 2 or 3 amino acids into an amino acid sequence consisting of SEQ ID NOS; 1-26 or the other amino acid sequences disclosed herein.

Another aspect of the disclosed technology is directed to use of a peptide or covalently modified peptide as described herein for the manufacture of a medicament, preferably a vaccine for the treatment or prevention of cancer. Such a use may be directed to manufacture of a. medicament to treat a cancer that expresses PRAME, or a cancer selected from the group consisting of melanoma, lymphoma, papillomas, breast or cervical carcinomas, acute and chronic leukemia, medulloblastoma, non-small cell lung carcinoma, head and neck cancer, renal carcinoma, pancreatic carcinoma, prostate cancer, small cell lung cancer, multiple myeloma., sarcomas and hematological malignancies like chronic myeloid leukemia and acute myeloid leukemia.

Another aspect of the disclosure is directed to a composition comprising the peptide or covalently-modified peptide as disclosed above, such as a peptide comprising, consisting essentially of, or consisting of SEQ ID NOS; 1-26, and a pharmaceutically acceptable adjuvant, carrier, or excipient.

In some embodiments a peptide epitope as disclosed herein is complexed with a HLA class 1 or HLA class 2 antigen.

In some embodiments, such a composition may comprise 1, 2, 3, 4, 5, 6, 7, 8, 9, 10 or more of the peptides disclosed herein, or peptides or antigens from other cancers. In such a composition the peptide or covalently-modified peptide may have a length of no more than 10, 15, 20, 25, 30, 35, 40, 45, 50, 55, 60, 65, 70 or >70 contiguous amino acid residues.

Such a composition may comprise 1, 3, 4, 5, 6, 7, 8, 9, 10 or more peptides of SEQ ID NOS: 1-26.

The composition may further comprise an adjuvant or be formulated as a peptide-based vaccine. Thus, a further aspect of the invention relates to an immunogen or vaccine comprising the peptide epitopes of SEQ ID NOS; 1-26 described herein, and, optionally a suitable excipient and/or adjuvant. In one embodiment a peptide or modified PR ME peptide, such as those comprising a sequence of SEQ ID NOS; 1-26 may be bound to an immunogenic carrier such as BSA, KLH, tetanus toxoid or other immunogenic carrier; or may be incorporated into a liposome.

A liposome may be formulated to contain lipid A, muramyldipeptide or IL-1 as immunomodulators. Types and formulations of liposomes suitable for carriers of immunogens are known in the art and are incorporated by reference to Kaskin, KP, et al., UKR BIOKHIM ZH (59(4):100-107 (1978) and to Chapter 4, Liposonlat-based therapeutic carriers for vaccine and gene delivery, M. Rahman, et al,, NANOTECHNOLOGY-BASED APPROACHES FOR TARGETING AND DELIVERY OF DRUGS AND GENES, 2017, Pages 151-166.

In general, a peptide or modified peptide as described herein may be incorporated into a composition. Typically, such a composition will include a pharmaceutically acceptable excipient or carrier and may further contain an adjuvant or other active agent.

The term carrier encompasses any excipient, binder, diluent, filler, salt, buffer, solubilizer, lipid, stabilizer, or other material well known in the art for use in pharmaceutical formulations, for example, for intravenous administration a carrier may be sodium chloride 0.9% or mixtures of normal saline with glucose or mannose. The choice of a carrier for use in a composition will depend upon the intended route of administration for the composition. The preparation of pharmaceutically acceptable carriers and formulations containing these materials is described in, e.g., Remington Pharmaceutical Sciences, 21st Edition, ed. University of the Sciences in Philadelphia, Lippincott, Williams & Wilkins, Philadelphia Pa., 2005, which is incorporated herein by reference in its entirety.

An adjuvant is a pharmacological or agent that modifies the effect of other agents. Adjuvants may be added to the materials disclosed herein, such as peptides, peptide constructs, cells and nucleic acids to boost the humoral or cellular immune responses and produce more intense or longer-lasting immunity, thus minimizing the dose of material needed.

Adjuvants that may be compounded with, or otherwise used along with the PRAME, peptide epitopes, modified peptides, peptide constructs, cells expressing PRAME, or nucleic acid encoding PRAME, such as those encoding peptide epitopes comprising SEQ ID NOS: 1.-26, Adjuvants include, but are not limited to, inorganic compounds including alum, aluminum hydroxide, aluminum phosphate, calcium phosphate hydroxide; mineral oil or paraffin oil; bacterial products or their im. ologically active fractions, such as those derived killed Bordetella pertussis, Mycobacterium bovis, or bacterial toxoids; organics such as squalene; detergents such as Quil A, saponins such as Quillaja, soybean or polygala senega; cytokines such as IL-1, IL-2 or IL-12; Freunds complete adjuvant or Freunds incomplete adjuvant; and food based oils like Adjuvant 65, which is a product based on peanut oil. Those skilled in the medical or immunological arts may select an appropriate adjuvant based on the type of patient and mode of administration of the materials described herein.

For therapeutic purposes, formulations for parenteral administration of a peptide composition can be in the form of aqueous or non-aqueous isotonic sterile injection solutions or suspensions. The term parenteral, as used herein, includes intravenous, intravesical, intraperitoneal, subcutaneous, intramuscular, intralesional, intracranial, intrapulmonal, intracardial, intrasternal, and sublingual injections, or infusion techniques. These solutions and suspensions can be prepared from sterile powders or granules having one or more of the carriers or diluents mentioned for use in the formulations for oral administration, preferably in a digestion-resistant form such as an enteric coating. The active ingredient can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Injectable preparations of the PRAME peptide epitopes described herein, for example, sterile injectable aqueous or oleaginous suspensions can be formulated according to the known art using suitable dispersing or wetting ingredients and suspending ingredients. The sterile injectable preparation can also be a sterile injectable solution or suspension in a non-toxic parenterally acceptable diluent or solvent, for example, as a solution in 1,3-butanediol. Among the acceptable vehicles and solvents that can be employed are water, Ringers solution, and isotonic sodium chloride solution. In addition, sterile, fixed oils are conventionally employed as a solvent or suspending medium. For this purpose any bland fixed oil can be employed including synthetic mono- or diglycerides. In addition, fatty acids, such as oleic acid, find use in the preparation of injectables. Dimethyl acetamide, surfactants including ionic and non-ionic detergents, polyethylene glycols can be used. Mixtures of solvents and wetting ingredients such as those discussed above are also useful.

Another aspect of the disclosure is directed to a method for treating a subject having cancer expressing PRAME comprising administering a composition comprising a peptide or covalently modified peptide as described herein to a subject having a cancer that expresses PRAME.

In some embodiments, the composition is administered in combination with antigen presenting cells which restrict said peptide by an HLA class 1 or HLA class 2 antigen shared with the subject.

Another aspect of the disclosure is directed to an artificial polynucleotide construct, which may be DNA, RNA or a modified DNA or RNA, that encodes a peptide comprising an amino acid sequence of SEQ ID NOS; 1-26. wherein said encoded amino acid sequence is no longer than 10, 15, 20, 25, 30, 35, 40, 45 or 50 contiguous amino acid residues. This construct may be incorporated into a vector or into the nucleic acids of a host cell.

Another embodiment of this technology is a cell comprising the artificial polynucleotide construct described above that expresses at least one HLA class 1 or HLA class 2 antigen shared restricts the peptide encoded by said artificial polynucleotide construct.

Another aspect of the disclosures is the use of an artificial polynucleotide construct as described above for the manufacture of a medicament, preferably for manufacture of a nucleic acid based vaccine for the treatment or prevention of cancer. In some embodiments of this use, the cancer expresses PRAME and is selected from the group consisting of melanoma, lymphoma, papillomas, breast or cervical carcinomas, acute and chronic leukemia, medulloblastoma, non-small cell lung carcinoma, head and neck cancer, renal carcinoma, pancreatic carcinoma, prostate cancer, small cell lung cancer, multiple myeloma, sarcomas and hematological malignancies like chronic myeloid leukemia and acute myeloid leukemia.

Another aspect of the invention is directed to a method or a use of the peptides, such as those comprising or consisting of SEQ. ID NOS: 1-26, for detection of T cells recognizing PRAME epitopes. In such as a method the activation of cells such as PBMCs from a patient in response to a peptide or modified peptide as disclosed herein in comparison to control cells, such as cells contacted with a non-PRAME peptide or control peptide, indicate the presence of PRAME-specific cells in the subject. Another embodiment of the invention comprises a kit for detecting T cells which recognize PRAME comprising one or more peptides described by SEQ ID NOS: 1-26and optionally, fluorophore-conjugated antibodies to CD4, CD8, TCRαβ, CXCR3, CXCR5, CCR6, CD127, CD25, CD56 or other T cell surface markers. It may also include other components of an IFN-γ, ELIS spot assay. Other kit components and methods of detection of PRAINTE specific T cells are known in the art and are incorporated by reference to Phetsouphanh C, et al, INT J MOL SCI . 2015 Aug. 12;16(8):18878-93. doi: 10.3390/ijms160818878.

Further aspects and description of the disclosure include the following.

Neoplasm. A neoplasm or tumor is a group of cells that have undergone unregulated growth and will often form a mass or lump, but may be distributed diffusely Cancerous cells are one type of neoplasm as are benign tumors, The methods and products described herein may be used to treat neoplasms that express PRAME.

Cancer. This term refers to a large family of diseases that involve abnormal cell growth which usually have the potential to invade or spread to other parts of the body. This term encompasses both solid cancers (such as solid tumors) and liquid cancers (such as leukemia and other blood cancers). Cancer cells typically manifest one or more of the following characteristics: cell growth and division in the absence of normal physiological signals, continuous growth and cell division in the presence of normal inhibitory signals, reduction or avoidance of programmed cell death (apoptosis), enhanced or unlimited capacity to divide compared to normal cells, promotion of blood vessel construction (angiogenesis), and/or invasion of tissues and formation of metastases. The methods and products described herein may be used to treat cancers that express PRAME.

T cells A T cell is a type of lymphocyte, which develops in the thymus gland and plays a central role in the immune response. T cells can be distinguished from other lymphocytes by the presence of a I-cell receptor on the cell surface. These immune cells originate as precursor cells, derived from bone marrow, and develop into several distinct types of T cells once they have emigrated to the -thymus gland, T cell differentiation continues even after they have left the thymus. FIG. 8 describes subtypes of T cells.

Groups of specific, differentiated T cells have an important role in controlling and shaping the immune response by providing a variety of immune-related functions.

One of these functions is immune-mediated cell death, and it is carried out by T cells in several ways: CD8⁺ T cells, also known as “killer cells”, are cytotoxic this means that they are able to directly kill virus-infected cells as well as cancer cells. CD8⁺ T cells are also able to utilize small signaling proteins, known as cytokines, to ecr it other cells when mounting an immune response.

A different population of T cells, the CD4⁺ T cells, function as “helper cells”. Unlike CD8⁺ killer T cells, these CD4⁺ helper T cells function by indirectly killing cells identified as foreign: they determine if and how other parts of the immune system respond to a specific. perceived threat, Helper T cells also use cytokine signaling to influence regulatory B cells directly, and other cell populations indirectly.

Regulatory T cells are yet another distinct population of these cells that provide the critical mechanism of tolerance, whereby immune cells are able to distinguish invading cells from “self”thus preventing immune cells from inappropriately mounting a response against oneself (which would by definition be an “autoimmune”response). For this reason these regulatory T cells have also been called “suppressor”T cells. These same self-tolerant cells are co-opted by cancer cells to prevent the recognition of, and an immune response against, tumor cells.

Subpopulations of T cells which may be separated or enriched and used in the methods disclosed herein include those described by FIG. 8.

Naïve T cell. This term describes T cells which have: not yet encountered their specific antigen. In peripheral lymphoid organs naïve T lymphocytes can interact with antigen presenting cells (APCs), which use an MHC molecule to present antigen. If the T lymphocyte recognizes a specific antigen, it will proliferate and differentiate into effector T lymphocytes of a particular type. in contrast, a subject who is naive to PRAM E includes one who has not developed a neoplasm or cancer expressing PRAME and whose immune system is tolerant to or does not substantially recognize PRAME in normal tissues, such as in testicular tissue.

Precursor T cell, This term describes cells which can differentiate or be induced to differentiate into T cells. It includes multipotential hematopoietic stem cells (hemocytoblasts), common lymphoid progenitors; and small lymphocytes.

The term “isolated”means separated from components in which a material is ordinarily associated, for example, an isolated PBMC population can be separated from red blood cells, plasma, and other components of blood and an isolated T cell can be separated or substantially separated from other types of leukocytes.

A “control”is a reference sample of subject used for purposes of comparison with a test sample or test subject, Positive controls measure an expected response and negative controls provided reference points for samples where no response is expected.

“Cord blood”has its normal meaning in the art and refers to blood that remains in the placenta and umbilical cord after birth and contains hematopoietic stern cell. Cord blood may be fresh, cryopreserved, or obtained from a cord blood bank, It is one source of cells that can be HLA-matched to a subject or patient for production of PRAME specific T cells.

PRAME. The tumor-associated antigen PRAME (PReferentially expressed Antigen in MElanorna cells) was originally identified as an antigen recognized by cytotoxic T lymphocytes capable of lysing melanoma cells (Ikeda et al., Immunity. 1997; 6:199-208.) PRAME is a cancer-testis antigen overexpressed in a variety of human malignancies, including lymphoid and myeloid malignancies and solid tumors, while being poorly expressed in healthy adult tissues except for testis, endometrium and at very low levels in ovaries and adrenal glands.

PRAME is expressed in about 90% among melar oma subtypes while negative in about 85% of cutaneous melanocytic nevi. It is not expressed in normal tissues, except testis. This expression pattern is similar .o that of other cancer/testes (CT) antigens, such as MAGE, BAGE and GAGE.

PRAME is also highly expressed in a wide range of non-melanoma cancers including leukemia, sarcoma, renal cell cancer, Wilms tumor, non-small cell lung cancer (NSCLC), neuroblastoma, breast cancer, and multiple myeloma. and PRAME expression has been associated with poor prognosis in a multitude of solid tumors. PRAME expression is minimal in. healthy tissues such as the gonads, adrenal glands, bone marrow, and brain with highest expression in the testes.

Various isoforms of PRAME are recognized, the sequence of one isoforms is given. below. The methods disclosed herein may be practiced with other isoforms of PRAME which comprise the same or immunologically similar epitopes to those disclosed herein. Similar peptide epitopes may bind the same HLA class 1 or class 2 molecules as the corresponding peptide epitopes described herein (such as those of SEQ ID NOS; 1-26) acid can be recognized by the same T cells as those recognizing the peptide epitopes described herein.

PRAME polynucleotide sequence. The polynucleotide sequence of the PRAME gene and the amino acid sequences of its isoforms are described by, and incorporated by reference to, hypertext transfer protocol secureihmw.uniprot.orgluniprot/P78395 (last accessed May 27, 2021). The amino acid sequence of one PRAME isoform (P78395-1 [UniParc]FASTA], incorporated by reference) is given below and by SEQ ID NO: 28.

```
                10 20 30 40
MERRRLWGSI QSRYISMSVW TSPRRLVELA GQSLLKDEAL
                50 60 70 80
AIAALELLPR ELFPPLFMAA FDGRHSQTLK AMVQAWPFTC
                90 100 110 120
LPLGVLMKGQ HLHLETFKAV LDGLDVLLAQ EVRPRRWKLQ
              130 140 150 160
VLDLRKNSHQ DFWTVWSGNR ASLYSFPEPE AAQPMTKKRK
              170 180 190 200
VDGLSTEAEQ PFIPVEVLVD LFLKEGACDE LFSYLIEKVK
              210 220 230 240
RKKNVLRLCC KKLKIFAMPM QDIKMILKMV QLDSIEDLEV
              250 260 270 280
TCTWKLPTLA KFSPYLGQMI NLRRLLLSHI HASSYISPEK
              290 300 310 320
EEQYIAQFTS QFLSLQCLQA LYVDSLFFLR GRLDQLLRHV
              330 340 350 360
MNPLETLSIT NCRLSEGDVM HLSQSPSVSQ LSVLSLSGVM
              370 380 390 400
LTDVSPEPLQ ALLERASATL QDLVFDECGI TDDQLLALLP
              410 420 430 440
SLSHCSQLTT LSFYGNSISI SALQSLLQHL IGLSNLTHVL
              450 460 470 480
YPVPLESYED IHGTLHLERL AYLHARLREL LCELGRPSMV
              490 500
WLSANPCPHC GDRTFYDPEP ILCPCFMPN
```

Peptide epitopes of PRAME These include peptides having unmodified amino acid sequences which correspond to fragments of a longer PRAME amino acid sequence that when presented by an HLA class 1 or HLA class 2 molecules are recognized by T cells. Additionally, this term encompasses modified peptides, such as peptides with modified C or N terminal residues, modified amino acid side-chains, or other modified peptides disclosed herein, which are HLA restrictable and which are recognized by T cells which also recognize a corresponding unmodified amino acid sequence. Peptide epitopes of PRAME may also be present on longer peptides comprising the amino acid sequences of SEQ ED NOS: 1-26, such as peptides having a length up to 15, 20, 25, 30, 35, 40, 45, 50 or more amino acid residues and which can be processed and restricted by, or directly restricted by or bound to, HLA class 1 or HLA class 2 molecules. In some embodiments a PRAME HLA class 1 restricted peptide epitope will consist of 8, 9 or 10 contiguous residues of PRAME and a PRAME HLA class 2 restricted peptide epitope will consist of 13, 14, 15, 16, 17 or 18 contiguous residues of PRAME. Longer peptides may be internalized by an antigen presenting cell and processed into shorter peptides coniprising a PRAME epitope that can complexwith class 1 or class 2 molecules. Peptide epitopes also include truncated versions of the peptides consisting of the amino acid sequences of SEQ ID NOS: 1-26which retain a capacity to be HLA class 1 or HLA class 2 restricted and recognized by T cells.

As disclosed herein, HLA-restricted PRAME. peptide epitopes have been identified. These epitopes include those restricted to HLA-A*02, a common HLA type especially among the general Caucasian population, as well as epitopes restricted by HLA types that are prevalent among other ethnic groups. These epitopes find utility for off-the shelf T-cell therapy and anti-tumor vaccines.

HLA haplotypes. Cof mon HLA haplotypes are described by, and incorporated by reference to, Pedron, B., et a/. Common genomic HLA haplotypes contributing to successful donor search in uwe Sated hematopoietic transplantation BONE MARROW TRANSPLANT 31, 423-427 (2003). https://doi.org/10.1038/sj.bmt.1703876; Maiers, M., et al., High resolution HLA alleles and haplotypes in the US population. HUMAN IMMUNOLOGY, 2007, 68, 779-788; and to Hurley, C. K. et al., Common, intermediate and well-docuine ie HLA alleles i world populations.version 3.0,0., HLA RESP, GEN. 2020, 95(60: 503-637. Some common HLA haplotypes include HLA-A1, HLA-A2, HLA-A3, HLA-A68, HLA-137, HLA-B8, HLA-B35, HLA-B44, HLA-B60, HLA-B61 and HLA-B62.

The HLA alleles described herein are expressed in codominant fashion. This means the alleles (variants) inherited from both parents are expressed equally. Each person carries 2 alleles of each of the 3 class-I genes, (H11.4-A. HLA-B and 1114-C), and so can express six different types of HLA class I molecules or antigens.

In the HLA class II locus, each person inherits a pair of HLA-DP genes (DPA1 and DPB1, which encode α and β chains), a couple of genes HLA-DQ (DQA1 and DQB1, for α and β chains), one gene HLA-DRα (DRA1), and one or more genes HLA-DRβ (DRB1 and DRB3, -4 or -5). That means that one heterozygous individual can inherit six or eight functioning class II alleles, three or more from each parent, The role of DQA2 or DQB2 is not verified. The DRB2, DRB6, DRB7, DRB8 and DRB9 are pseudogenes.

The PRAME peptides disclosed herein will bind to one or more of the HLA class 1 or class 2 MHC molecules described herein. There are two types of HLA molecules, class 1 and class 2, and both are highly polymorphic. The core binding subsequence of both HLA class 1 and 2 is approximately 9 amino acids long. However, HLA class 1 molecules rarely bind peptides much longer than 9 amino acids, while HLA class 2 molecules can accommodate longer peptides of 10-30 residues. One skilled in the immunological arts may select a peptide antigen length suitable for binding to HLA class 1 or class 2 molecules.

In some embodiments, the HLA class 1 or class 2 restricted PRANTE peptides disclosed herein, or shorter fragments thereof, may range in length from 6, 7, 8, 9, 10, 11, 12, 13, 14, 15, 16, 17, 18, 19, 20, 21, 23, 24, 25, 26, 27, 28, 29 or 30 amino acid residues. In some embodiments, a peptide epitope, such as those comprising SEQ ID NOS; 1-26, may be processed. by an antigen presenting cells prior to its association with an HLA class 1 or class 2 molecule. Such processing may decrease its peptide length. Antigen processing and presenting machinery and mechanisms processing class 1 presented peptides (such as those presented by HLA class 1 molecules), is known in the art and incorporated by reference to Leoni, P. et al., AMC Class I Antigen Processing and Presenting Machinery: Organization, Function, and Defects in Tumor Cells, J. Nat. Cancer Inst. 105(11.6): 1172-1187; and to Roche, P. A., et al., The ins and outs of MHC class II-mediated antigen processing and presentation, NATURE REVIEWS, 2015, 15, 203-216. Antigen-presenting cells (APC), including B cells and dendritic cells, present the peptides to cytotoxic T cells. Peptides (regardless of length) can be presented by virtually any cell type as they require minimal processing by endonucleases to produce the 8-10 mer peptides required to be presented by HLA class I molecules expressed by the APC.

HLA Matching. A subject may be matched to a donor at least one HLA haplotype (e.g., HLA-A1), HLA allele or synonymous allele (e.g., HLA-A*02, :HLA-B*07), or by common expression of at least one specific HLA protein (e.g., HLA-A*02:101, HLA-B*0701). Preferred matching occurs at the level of an allele group.

Matching may be performed by methods known in the art which include genetic or serological procedures to determine whether the donor and subject share an HLA allele, allele group, or specific HLA protein. PCR (polymerase chain reaction) and NGS (next generation sequencing) HLA typing methods are known and commercially available for HLA genotyping the HLA class I and class lI gene polymorphisms for an individual, for example, from CD Genomics (hypertext transfer protocol secure: //www.cd-genomics.com/Genotyping.html) and others.

In some embodiments, a donor may be a close family member, such as a parent, sibling son or daughter, uncle or aunt, grandparent, cousin, who shares with a recipient, an appropriate HLA class 1 or class 2 molecule that restricts a peptide epitope of PRAME.

HLA class I and HLA class 2 antigen processing and presentation. HLA (human leukocyte antigens) are major histocompatibility (MHC) molecules.

HLA class 1 molecules comprise a polymorphic alpha chain and beta.-2 microglobulin which forms a complex when a peptide, such as a peptide epitope of SEQ ID NOS; 1-26, binds to the alpha chain. All nucleated cells express HLA class 1 molecules. Cytotoxic (CDS) T cells are able to respond to an HLA class I peptide complex. In nature, peptides presented by HLA class 1 molecules are typically generated by the cytosolic proteasome and loaded on a class 1 molecule in the endoplasmic reticulum. Usually, HLA class 1 molecules or complexes bind to peptide antigens ranging in length from 8-10 amino acid residues. In some embodiments, antigen presenting cells may be pulsed with peptides that directly bind to HLA class 1 molecules and form a HLA-peptide complex or, alternatively, are internalized, processed and presented as part of an HLA-peptide complex.

HLA class 2 molecules comprise polymorphic alpha and beta chains which together bind a peptide and form a complex recognizable by T helper (CD4⁺) cells. Dendritic cells, mononuclear phagocytes, some endothelial cells, and thymic epithelium express HLA class 2 molecules. In nature, peptides presented by HLA class 2 molecules are usually derived from proteins present in endosomes or lysosomes which often are internalized from the extracellular medium. Cellular proteases such as cathepsin generate peptides from these proteins which are presented by the HLA class 2 complex. Usually, HLA class 2 molecules or complexes bind to peptide antigens ranging in length from 13-18 amino acid residues. In some embodiments, antigen presenting cells may be pulsed with peptides that directly bind to HLA class 2 molecules and form a HLA-peptide complex or, alternatively, are internalized, processed and presented as part of an HLA-peptide complex.

Methods for producing T cells ex vivo that recognize tumor antigens such as PRAME are incorporated by reference to using the steps disclosed by U.S. Pat. Nos. 9,885,021, 10,934,525, or by PCT/US20161023413 which are incorporated by reference for all purposes. These cells may be produced using autologous donor cells (e.g., from a patient's own bone marrow or cord blood or using cells from a donor who shares 1, 2, 3, 4, 5, 6 or more MHC class l or class II molecules such as the HLA molecules mentioned above. T cells produced ex vivo to a PRAME peptide may be administered to a fully histocompatible (e.g., autologous, or identical twin) or partially histocompatible (e.g., someone who shares at least one HLA class 1 for class 2 allele or protein, but not all HLA alleles or proteins, with a donor).

Off the shelf T cells. These are ready-to-use, off-the-shelf therapeutic cells. These are produced using the PRAME epitopes described herein, usually from the blood cells from normal, healthy donors who are at least partially HLA matched to a subject undergoing treatment. Such off-the-shelf T cells are typically well characterized as to origin and HLA background and by an ability to kill cancer cells expressing PRAME. In many embodiments, the T cells are cryo-preserved—stored frozen in liquid nitrogen—until its time to use them. In one embodiment, a cancer patient visits a physician where cancer markers such as PRAME are identified and where the subjects HLA background is determined or referenced. With the identity of the cancer-specific or cancer-associated antigens and the subjects HLA background in hand, the physician visits a cell bank filled with large below zero freezers and selects a banked cell sample suitable for therapeutic use against the particular cancer in a subject having a particular HLA background. These ‘off-the-shelf’, ready-made cells are thawed, prepared or expanded and infused into the patient several days later to recognize and destroy cells with the patients cancer-specific markers such as PRAME. Development of an effective off-the-shelf adoptive T-cell therapy for patients with relapsed or refractory malignancies expressing PRAME antigen depends on the identification of PRAME antigens recognized by the tumor-associated antigen-T cell product. As disclosed herein the inventors have broadened the repertoire of known ,HLA-restricted PRAME peptides and their HLA-restricted alleles to facilitate the production and use off-the-shelf PRAME specific T-cells for a variety of patients including those pwith relapsed or refractory malignancies expressing PRAME antigen.

### EXAMPLE 1

Peptide libraries of 125 overlapping 15-mer peptides spanning the entire PRAM, protein sequence were used to identify HLA class 1 and class ll-restricted epitopes. We also determined the HLA-restriction of the identified epitopes. As shown below, PRAME-specific T-cell products were successfully generated from PBMCs of 12 healthy donors. Ex-vivo expanded T-cells were polyclonal, consisting of both CD4⁺ and CD8⁺ T-cells which elicited anti-tumor activity in vitro. Nine MHC class I-restricted PRAME epitopes were identified. Sixteen 15-mer peptide sequences were characterized and confirmed as CD4-restricted epitopes. These CD⁴⁺ and CD⁸⁺ HLA-restricted PRAM epitopes can be used to produce TAA T-cells that recognize a broad range of CD⁴⁺ and CD8⁺ HLA-restricted PRAME, epitopes. The specificity of such TAA-T cells can be customized to recognize particular PRAME epitopes presented by a subjects MI-IC molecules, and thus provide a customized, off-the-shelf therapy for subjects having or at risk of developing cancers expressing PRAME.

Hematopoietic samples. Healthy donor buffy coats were obtained from the Gulf Coast Regional Blood Center, Houston, Tex. Peripheral blood mononuclear cells (PBMCs) were isolated by density gradient centrifugation using Lymphoprep (STEMCELL Technologies, Cambridge, Mass.) and frozen down in cryopreservation medium [50% RPMI 1640 medium, 10% dimethyl sulfoxide (DMSO) and 40% fetal bovine serum (FBS)]. IIL typing of the healthy donor PBMCs was performed by The Sequencing Center, Fort Collins, Colo.

Generation of PRAME-specific T-cell products. PRAME-specific T-cell products were generated from total PBMCs by previously established protocol which is described by and incorporated by reference to Weber, G. et al. Generation of multi-leukemia antigen-specific T cells to enhance the graft-versus-leukemia effect after allogeneic stem cell transplant. LEUKEMIA, 2013;27(7):1538-47. RPMI (GE Healthcare, Logan, Utah), 10% human AB serum (Gemini BioProducts, West Sacramento, Calif.), and supplemented with 2 mM GlutaMax (Gibco, Grand Island, N.Y.). Second and third restimulations of T-cells were carried out weekly with PRAME peptide-pulsed, irradiated. DC at an effector-to-target ratio of 20:1. On day 28, cells were harvested and evaluated for antigen specificity and functionality.

Anti-IFN-gamma enzyme-linked immunospot assay (ELISpot assay). PRAME specificity, of each T-cell product was evaluated by stimulating expanded cells with PRAME PepMix (JPT Peptide Technology, Berlin, Germany) and measuring IFN-γ production by ELISpot assay. T-cells were plated at 1×10⁵ cells/well with no peptide, PRAME PepMix (200 ng/well.), actin PepMix (tile irrelevant antigen used as a negative control), and Staphylococcal enterotoxin B (SEB), a superantigen used as a positive control. Spot-forming cells (SFCs) were enumerated by Zellnet Consulting (Fort Lee, N.J.).

Cytotoxicity Assay. Cytolytic activity of the tumor antigen (PRAME)-specific T-cells versus the Wilms tumor cell lines Wit49 (donated by Kentsis Research Lab, Sloan Kettering Institute, New York, N.Y.) and 17.94 (DSMZ, Braunschweig, Germany) was tested in a calcein AM cytotoxicity assay. Target cells were resuspended in complete medium at a final concentration of 10⁶/mL and incubated with 10 μM calcein-AM (Thermo-Fisher, Waltham, Mass.) for 30 minutes at 37°C. in 5% CO₂. After 2 washes in complete medium, cells were resuspended at 10⁵ cells/mL. Partially HLA-matched healthy donor derived PRAME-specific T-cells versus T-cells with irrelevant specificity (i.e., non-specifie T-cells (NSTs)), were co-cultured with tumor cells at effector to target (E:T) ratios of 40:1, 20:1 and 10:1 and 5:1 in triplicate in 96-well round bottom plate (Corning, N.Y.). PRAME-specific T-cell and -NST [phytohaemmaglutinin (PHA) blasts or PBMCs] effector cells were plated at appropriate ratios and incubated at 37°C. in 5% CO2 for 4 hours. After incubation, 75 μL of each supernatant was harvested and transferred into new plates. Samples were measured using a microplate spectrofluorometer (excitation filter: 485+9 nm; emission filter: 515+9 nm) with data expressed as arbitrary fluorescent units (AEU). Percent lysis was calculated according to the formula [(test release−spontaneous release)/(maximum release−spontaneous release)]×100. Spontaneous release represents calcein release from target cells in medium alone, and maximum release represents calcein release from target cells lysed in medium plus 2% Triton X-100, plated in triplicate at least. Background from media was subtracted from all the values.

immunophenotyping. PRAMSspecific T-cell products were phenotyped by extracellular antibody staining with anti- CD3. CD4, CD8, CD14, CD16, CD19, CD56, CD45RO, CD62L, CCR7 (Miltenyi Bit-Ace, Bergisch Gladbach, Germany; BioLegend, San Diego, Calif.) and acquired on a CytoFLEX cytometer (Beckman Coulter, Brea, Calif.). The data was analyzed using FlowIo X software (Flowk LLC, Ashland, Oreg.).

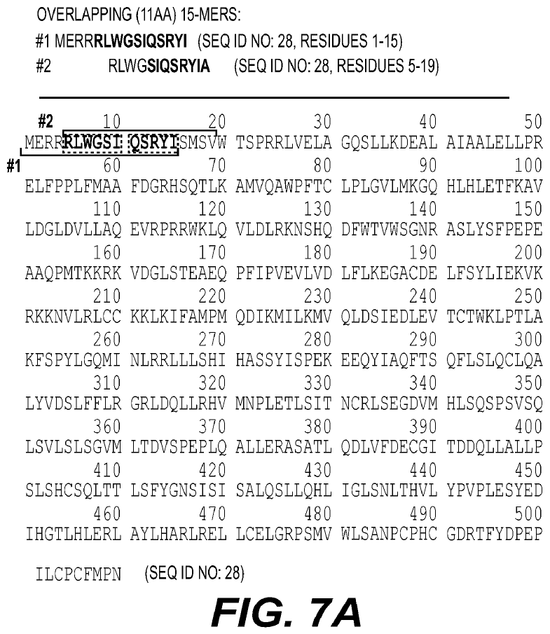

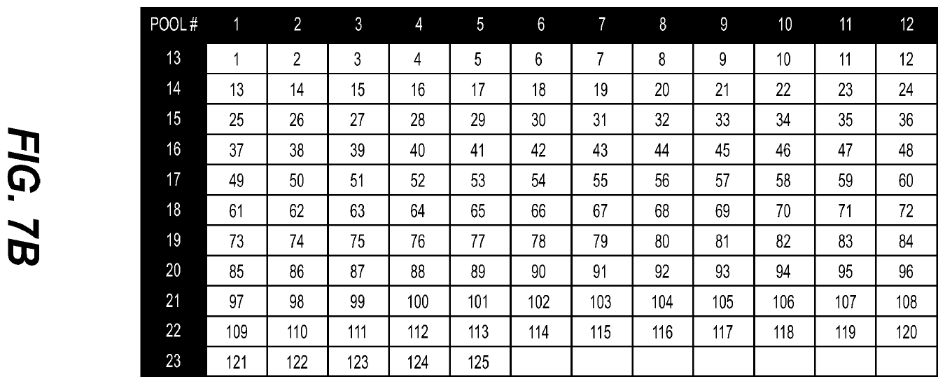

PRAME Peptides. The PRAME peptide library consisting of 125 15-mer peptides overlapping by 11 amino acids and spanning the entire sequence of PRAME protein was designed to identify HLA class 1 and HLA class H-restricted epitopes (A&A Labs, San Diego, Calif.) 23 peptide pools comprising 10-12 peptides were prepared so that each 15-mer peptide was included in only two pools (FIGS. 7A and 7B). To determine minimal PRAME epitopes, additional 9-mer peptides overlapping by 8 amino acids spanning immunogenic 15-mer peptides were obtained from A&A Labs, San Diego, Calif. All peptides were reconstituted at 10 μg/μl in DMSO and stored at −80°C. until further use.

Epitope mapping. T-cell response to pools of peptide libraries and individual PRAME peptides were determined. by IFN-γ ELISpot assay. ELISpot assay was performed as previously described. 1×10⁵ T-cells/well were plated alone, with actin (negative control), SEB (positive control), each peptide pool (1μg/well) and individual peptide (10 μg/well). IFN-γ spot-forming cells (SFC) were enumerated by Zellnet Consulting (Fort Lee, N.J.).

Responses that were at least 10 SFC/1×10⁵ T-cells or greater than two-fold the background level of nonstimulated T-cells or T-cells stimulated with actin were considered positive responses.

HLA restriction of individual peptides that showed specificity by ELISpot was determined by intracellular cytokine staining, measuring IFN-γ and TNF-α release. T-cells were stimulated for 6 h with PRAME PepMix or individual PRAME peptides (200 ng/peptide/well) in the presence of anti-CD28 and CD49d antibodies (RD Biosciences, San Jose, Calif., USA) and Brefeldin A (Golgiplug, BD Biosciences). Controls (unstimulated T-cells, actin, SEE) were included in each experiment. Intracellular IFN-γ (BioLegend, San Diego, Calif.) and TNF-α (Miltenyi Biotec, Bergisch Gladbach, Germany) staining were performed on fixed and permeabilized cells (Cytofix/Cytoperm, BD Biosciences). Data was acquired with a CytoFLEX cytometer (Beckman Coulter, Brea, Calif.), and analyzed using FlowJo Flow Cytometry software (FlowJo LLC, Ashland, Oreg.).

Minimal epitopes recognized by HLA I-restricted cell lines were determined by IFN-γ ELISpot assay. To confirm the restricted HLA allele, anti-human HLA class 1 B35 antigen monoclonal antibody was used in the ELISpot plate (MyBioSource, San Diego, Calif.).

Immuncyluoreseence. The coverslips were seeded with tumor cells and pancreatic cells at 2.5e⁵/500 μl per well and left overnight for the confluency to reach 50-70%. Once the confluency was achieved, the cells were rinsed in 1X PBS, followed by fixation with 4% PFA for 15 minutes at room temperature and permeabilization for 20 minutes in 0.1% Triton X-100.

Nonspecific binding was blocked with 2?BSA for an hour at room temperature, Primary anti-PRAME antibody (Sigma, St. Louis, Mo.) was diluted 1:30 in 0.1% BSA in PBS and incubated overnight at 4° C. After the overnight incubation, the slides were rinsed and incubated with an appropriate secondary antibody diluted 1:100 in 0.1% BSA in PBS for an hour at room temperature (Donkey Anti-Rabbit Alexa Flour 568, Abeam, Cambridge, Mass.). Prolong Gold Mounting media with DAPI was used to counterstain the nuclei and mount the slides. All images were taken 24 hours after mounting of slides and imaged using Olympus BX53 microscope.

Data analysis. Results were evaluated using descriptive statistics (medians and ranges). The Student t test was used to test for significance. Data analysis was performed in GraphPad Prism (GraphPad Software, La Jolla, Calif.).

PRAM mspeciic T-cells can be expanded from healthy donors. PRAME-specific T-cells were expanded from PBMCs obtained from twelve healthy donors. Priming and restimulations of PBMCs were carried out with PRAMS-pulsed, irradiated DC. A median of 452×10⁶ T-cells (range 161×10⁶−17.5×10⁸ T-cells) were harvested by day 21 of culture (FIG. IA). This showed that suitable numbers of PRAME-specific T cells can be expanded for clinical use or for off-the-shelf administration.

Phenotyping of expanded T-cells showed a median CD3⁺ content of 90.75% (range 83.9-97.8%) with a mixture ofCD4⁺ T-cells (median 39.6%, range 9.9-802%) and CD8⁺ T-cells (55.6%, 14.5-88.3%). There was no outgrowth of natural killer (NK) cells or natural killer T (N-KI) cells. B cells and dendritic cells accounted for less than 2% of final products, meeting clinical release criteria for TAA-T products for use in the clinic.

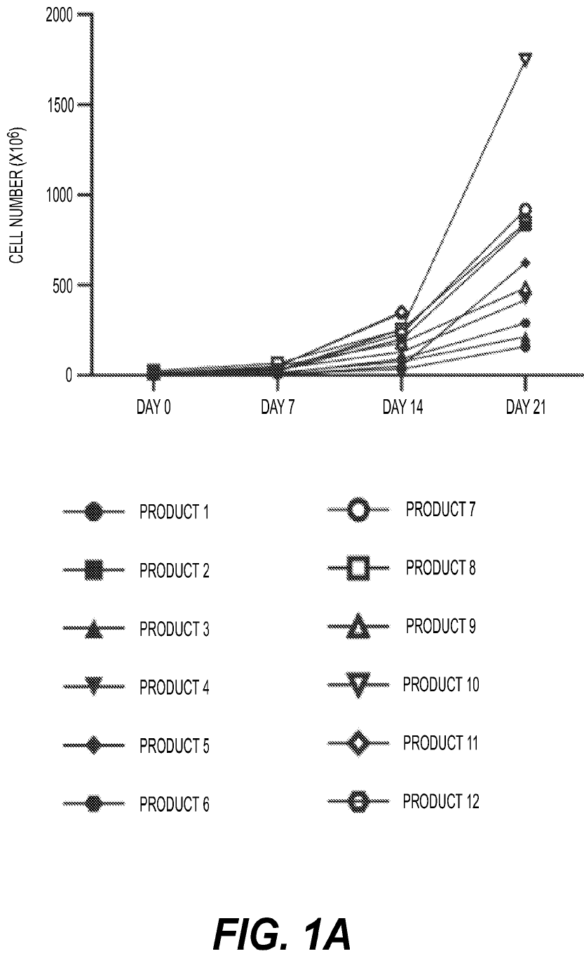

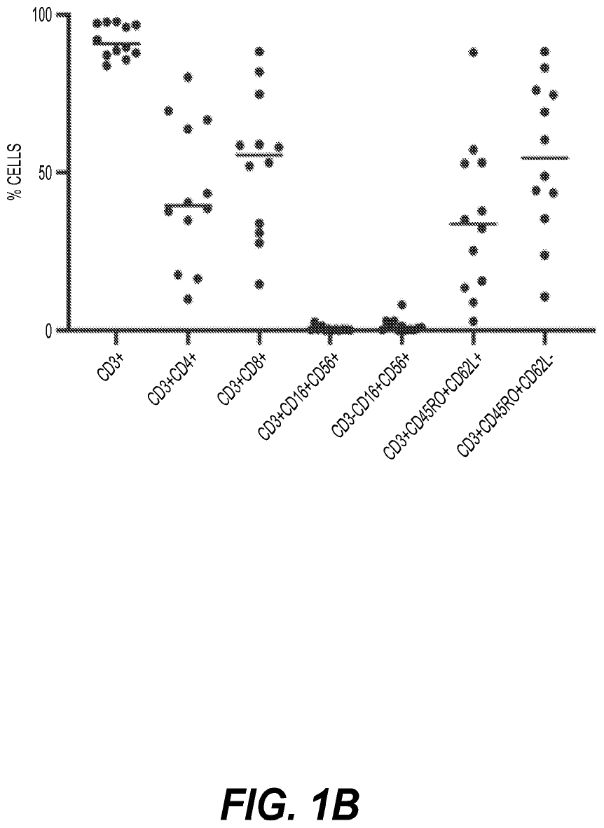

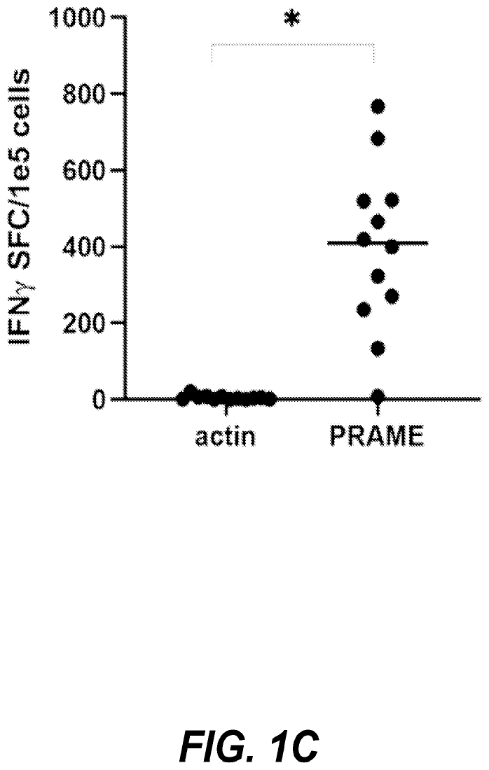

Expanded T-cells were composed of both central memory (median 33.7%, 2.88-88.0%) and effector memory (54.7%, 10.7-88.3%) T-cells (FIG. 1B).

PRAME antigen specificity was evaluated usinc, IFN-γ ELISpot assay. Eleven T-cell products demonstrated response to PRAME (median 405.48 SFC/1×10⁵ cells, range 8-762.5) while median actin (negative control) was 4.0 SFC/1×10⁵ cells (range 0.5-20) (p-value <0.0001) (FIG. 1C).

PRAME-specific T-cells elicit antitumor activity against partially HLA-matched solid tumor cell lines. To evaluate the in vitro anti-tumor activity of healthy donor-derived T-cells targeting PRAME, in a “third party setting”, T-cells were cocultured with the Wilms tumor cell line(s) Wit49 and WT 17.94 known to express PRAME (FIG. 2A).

Tumor cell lines were matched in at least one HLA-antigen (range 3-9) with the T-cell products (Table 3) which shows HLA types of Wilms tumor cell lines (17.94, Wit49) and partially matched TAA-T cell products.

TABLE S3
HLA matching of third-party TAA-T products

| | IMGT/ | IMGT/ | IMGT/ | IMGT/ | IMGT/ | IMGT/ | IMGT/ | IMGT/ | IMGT/ |
|---|---|---|---|---|---|---|---|---|---|
| | A | B | C | DPA1 | DPB1 | DQA1 | DQB1 | DRB1 | DRB3 |
| 17.94 | 1:01: | 8:01: | 7:01: | 1:03: | 04:01: | 05:01: | 02:01: | 03:01: | 01:01: |
| | 01 | 01 | 01 | 01 | 01 | 01 | 01 | 01 | 02 |
| Wit49 | 1:01: | | | 1:03: | 04:01: | | 02:01: | | |
| | 01 | | | 01 | 01 | | 01 | | |
| TAA-T | 1:01: | 8:01: | 7:01: | 1:03: | 04:01: | 05:01: | 02:01: | 03:01: | 01:01: |
| #1 | 01 | 01 | 01 | 01 | 01 | 01 | 01 | 01 | 02 |

| | IMGT/B | IMGT/C | IMGT/DPA1 | IMGT/DPB1 |
|---|---|---|---|---|
| 17.94 | | 7:01:01 | | |
| | 7:02:01 | 7:02:01 | 1:03:01 | 4:01:01 |
| TAA-T #2 | 7:02:01 | 7:01:01 | 1:03:01 | 4:01:01 |
| | | 7:02:01 | | |
| | IMGT/A | IMGT/DPA | IMGT/DPB1 | IMGT/DRB3 |
| TAA-T #3 | 2:01:01 | 1:03:01 | 4:01:01 | 1:01:02 |
| | 24:02:01 | | | |
| 17:94 | | 1:03:01 | 4:01:01 | 1:01:02 |
| | | 1:03:01 | 4:01:01 | |
| Wit49 | 24:02:01 | 1:03:01 | 4:01:01 | |

HLA class 1 (A, B, C) and HLA class II (DP, DQ, DR) groups were included in the analysis. Specifically, TAA-T was matched to 17.94 at 9 HI A. alleles and to Wit49 at 4 HLA alleles. TAA-T #2 was matched to 17,94 at 4 HLA alleles; but due to more limited TAA-T cell numbers, Wit49 was not included in the TAA-T #2 cytotoxicity assays. TAA-T #3 was matched to 17.94 and Wit49 at 3 HLA alleles each.

FIG. 2B-I shows the results of three TAA-T cell products which showed polyclonality, (FIG. 213) and specificity for PRAMS (FIG. 2C). These T-cell products were tested for cytolytic activity against the two Wilms tumor cell lines Wit49 and WT 17.94.

Specific tumor recognition and killing occurred even with single ITL A class I matched targets (FIGS. 2D-H).

As control for nonspecific lysis or allogeneic reactivity, T-cells with irrelevant specificity (Nonspecific T cells (INSTs)) from the same donor were used in all experiments and were not able to kill the tumor cells (FIGS. 2D-H). Moreover, TAA-T were not able to kill autologous PHA blasts/PBMCs (FIG. 2f) demonstrating a lack of autoreactivity in vitro.

As demonstrated, healthy donor-derived off-the-shelf PRAME-specific T-cells showed specific killing against PRAME tumor cell lines, with notable minimal cytotoxicity by NSTs derived from the same donor to these tumor cell lines and absence of autoreactivity in vitro, thereby demonstrating the potential for the use of these T-cell products in the off-the-shelf setting.

New CD8-restricted T-cell PRAME epitopes identified in healthy donor-derived T cell products. The inventors considered that in order to develop a third party PRAME-specific T cell bank, it would be important to identify epitopes recognized in the context of MHC class I and class II.

In order to map the class 1-restricted responses, a mapping grid consisting of 23 mini-pools comprising 125 individual 15-mer peptides spanning the entire PRAME protein sequence to identify PRAME epitopes eliciting a response by ex-vivo expanded T-cells was used (FIG. 7A). IFN-γ production by PRAME- sensitized T-cells in response to stimulation with mini-pools was measured by ELISpot assay.

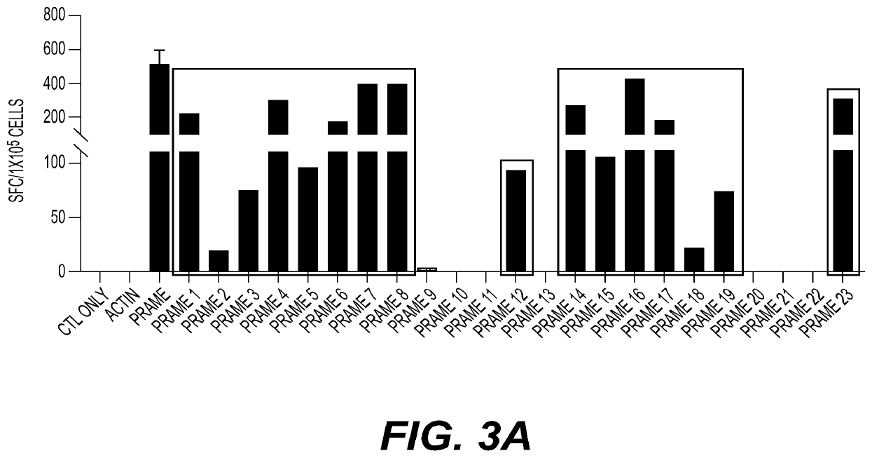

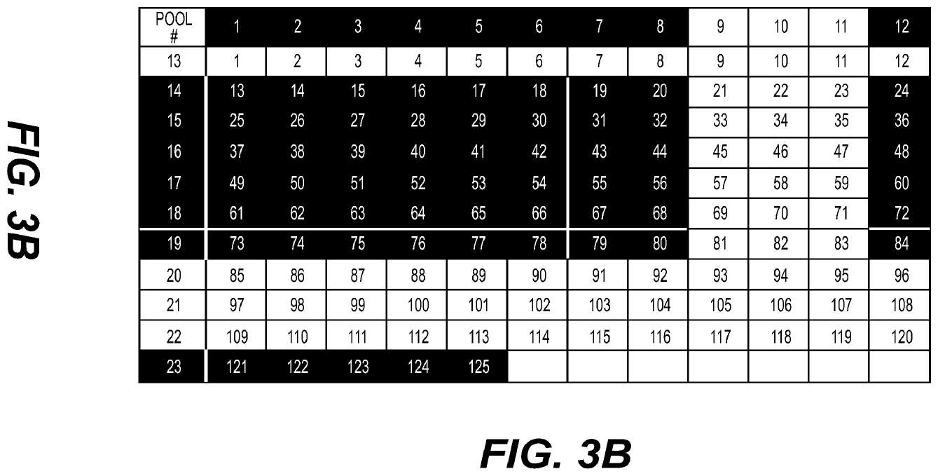

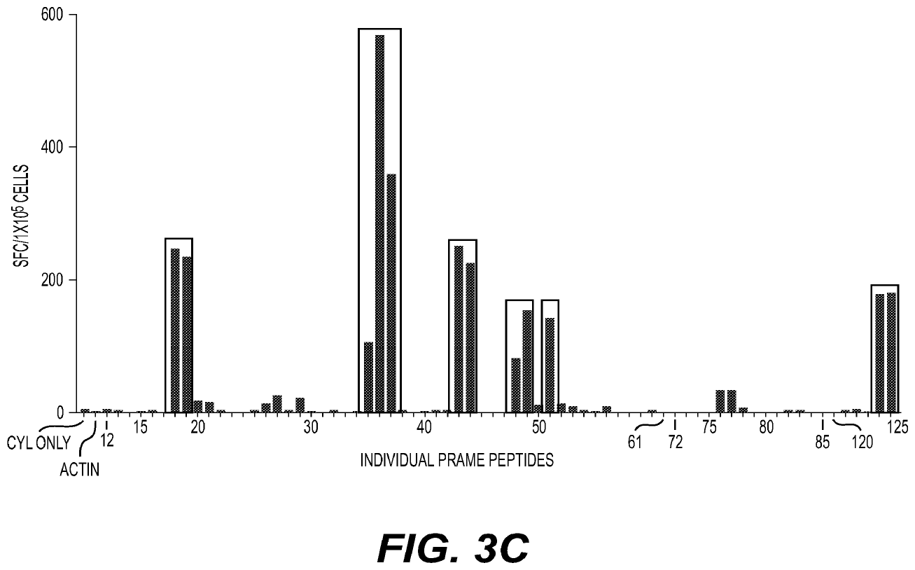

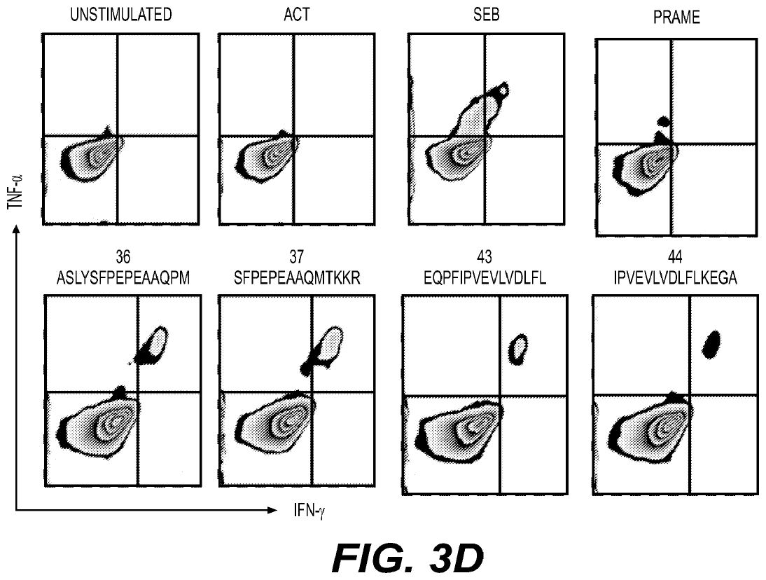

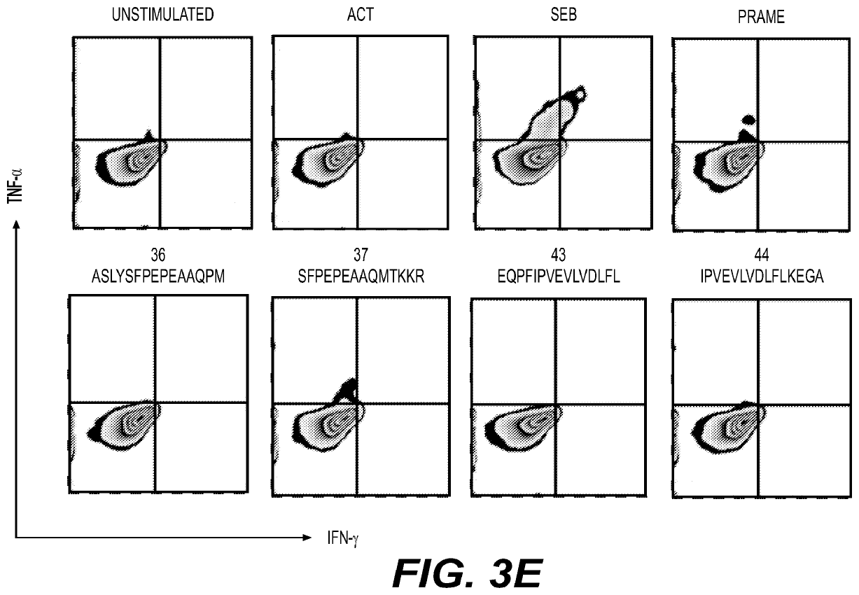

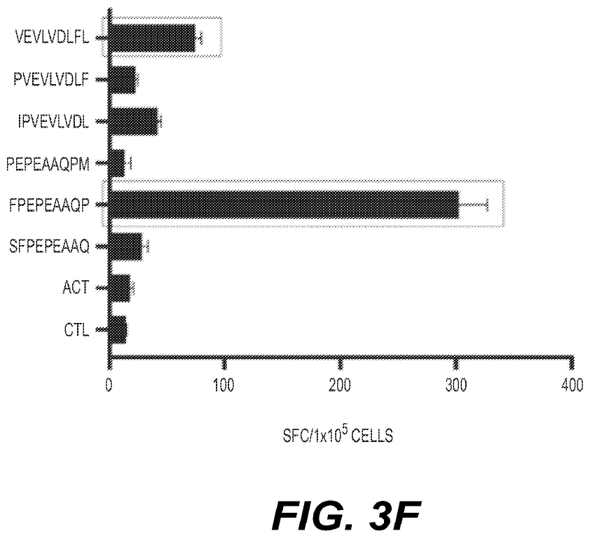

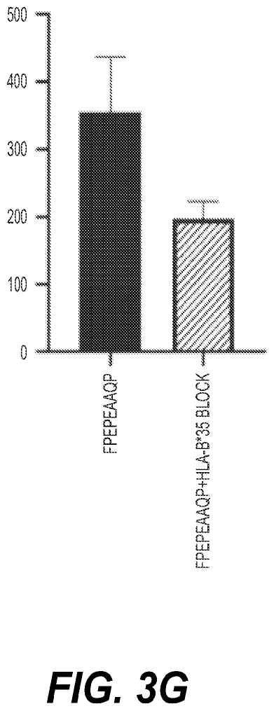

In an example of one T-cell product, dominant responses were observed for 16 mini-pools (1, 2, 3, 4, 5, 6, 7, 8, 12, 14, 15, 16, 17, 18, 19, and 23) (FIG. 3A).

T-cell responses to single 15-mer peptides selected from the grid and their neighboring peptides were determined by IFN-γ ELISpot assay (FIG. 3B).

Single peptides 18, 19, 35, 36, 37, 43, 44, 48, 49, 51, 124, and 125 were identified as immunogenic (FIG. 3C).

HLA restriction of these peptides was evaluated with intracellular IFN-γ and TNF-α cytokine staining.

As shown by FIG. 3D, CD8⁺ T-cells released IFN-γ and TNF-α in response to overlapping 15-mer peptides 36 ASIYSFPEPEAAQPM (SEQ ID NO: 30), 37 SFPEPEAAQPMTKKR (SEQ ID NO: 31), 43 EQPFIPVEVINDLFL (SEQ ID NO: 32), and 44 IPVEVINDLFLKEGA (SEQ ID NO: 33), indicating that these peptides were HLA class I-restricted epitopes (FIG. 3D).

As shown by FIG. 3E, CD4⁺ T-cells did not release IFN-γ and INF-α in response to these peptides.

The minimal 9-mer epitopes overlapping by 8 amino acids spanning the 15-mer peptides were determined by IFN-γ ELISpot.

As shown by FIG. 3F, CD8⁺ T-cells secreted IFN-γ upon stimulation with epitopes FPEPEAAQP (SEQ. ID NO: 6) and VEVLVDLFL (SEQ ID No: 7),

An algorithm (NetMHC [world wide web.cbs.dtu.dkiservicesiNetMFICpan/l] was applied to determine the restricted HLA allele of FPEPEAAQP (SEQ ID NO: 6) and VEVLVDLFL-FL: (SEQ ID NO: 7) epitopes.

The algorithm predicted strong binding to HLA-B*35:03 for FPEPEAAQP (SEQ ID NO: 6) and weak binding for 11.1A-B*38:01 for VEVLVDLFL, (SEQ ID NO: 7).

To confirm the HLA-B*35 restriction of FPEPEAAQP (SEQ ID NO: 6) further testing was performed using an anti-KLA-B*35 antibody.

T-cell product alone showed specificity for FPEPEAAQP (SEQ. ID NO: 6) while products with anti-HLA-B-*35 showed decreased specificity (FIG. 3G).

The complete data of novel CD⁸⁺-restricted T-cell epitopes identified in PRAME is summarized in Table 1. Predicted strong binding is shown in bold and weak binding is underlined.

TABLE 1
Peptide sequences of CD8-restricted
T-cell epitopes identified in PRAME

| | SEQ | Amino | | | |
|---|---|---|---|---|---|
| Peptide | ID | acid | | | |
| sequence | NO: | location | HLA-A | HLA-B | HLA-C |
| AGQSLLKDE | 1 | 29-37 | 01:01; 01:01 | 08:01; 18:01 | 06:02; 07:01 |
| AWPFTCLPL | 2 | 75-83 | 24:02; 26:01 | 15:08; 44:03 | 01:02; 16:01 |
| WPFTCLPLG | 3 | 76-84 | 24:02; 26:01 | 15:08; 44:03 | 01:02; 16:01 |
| SGNRASLYS | 4 | 137-145 | 02:01; 24:02 | 35:01; 35:03 | 04:01; 04:01 |
| LYSFPEPEA* | 5 | 143-151 | 02:01; 68:01 | 38:01; 44:02 | 05:01; 12:03 |
| FPEPEAAQP | 6 | 146-154 | 24:357; 26:01 | 35:03; 38:01 | 04:01; 12:03 |
| VEVLVDLFL | 7 | 175-183 | 24:357; 26:01 | 35:03; 38:01 | 04:01; 12:03 |
| EKVKRKKNV | 8 | 197-205 | 01:01; 01:01 | 08:01; 18:01 | 06:02; 07:01 |
| KVKRKKNVL | 9 | 198-206 | 01:01; 01:01 | 08:01; 18:01 | 06:02; 07:01 |
| RKKNVLRLC | 10 | 201-269 | 02:01; 24:02 | 35:01; 35:03 | 04:01; 04:01 |
| NLTHVLYPV* | 11 | 435-443 | 02:01; 26:01 | 51:01; 51:01 | 05:01; 16:02 |

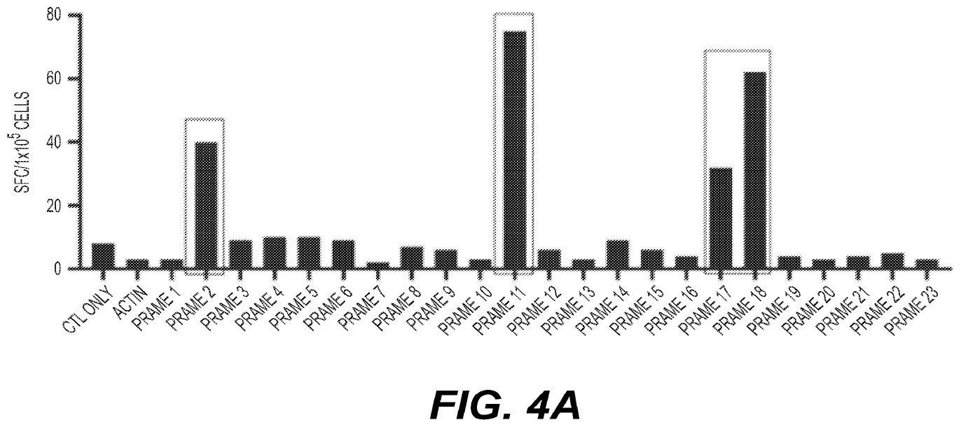

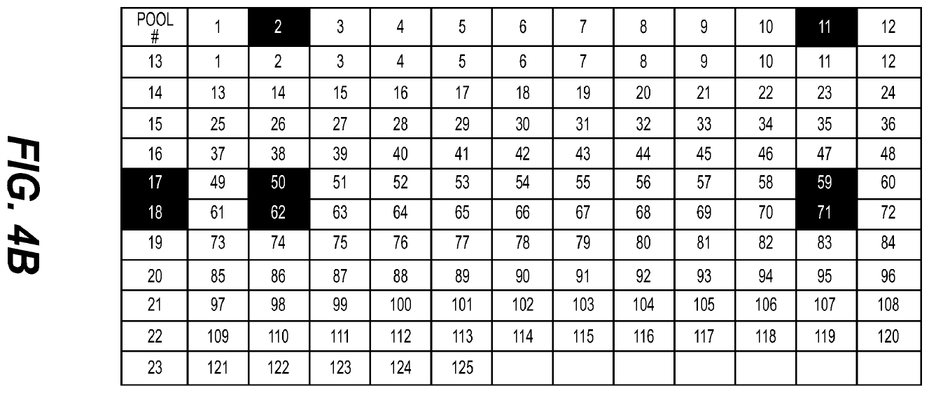

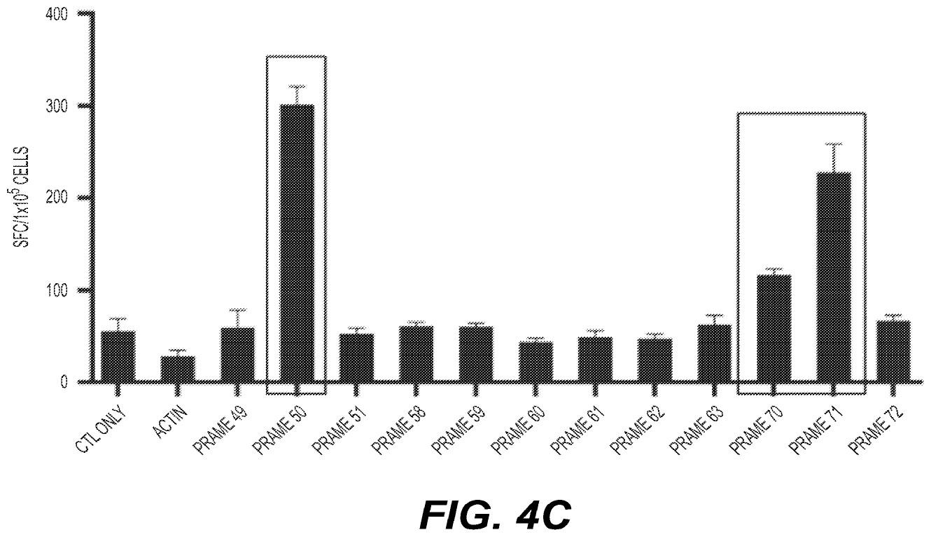

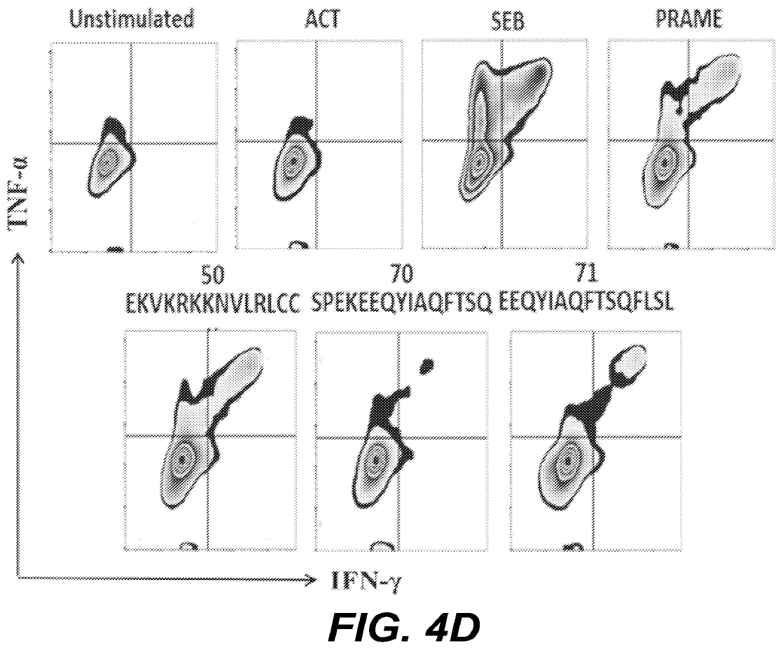

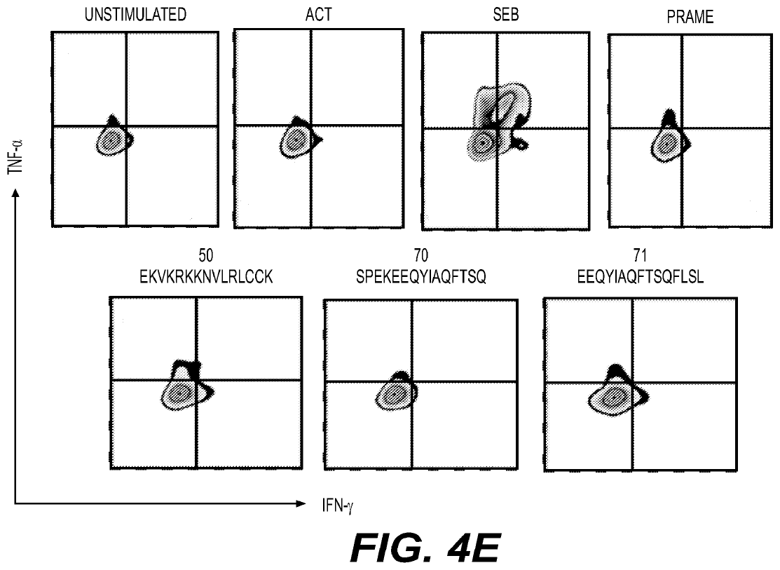

Broad CD4 specific activio) in donor PRAME-specific T-cells. The breadth of CD4-restricted epitopes recognized by PRA NTE-specific T-cells was analyzed using the same approach as above. A representative example is shown in FIGS. 4A-4E.

As shown by FIG. 4A, the T-cell product recognized 4 mini pools: 2, 11, 17, and 18.

As shown by FIG. 4B and 4C, testing of the single peptides and their neighboring peptides revealed recognition of single peptides 50 EKVKRKKNVLRLCCK (SEQ ID NO: 21), 70 SPEKEEQYIAQFTSQ (SEQ ID NO: 29), and 71 EEQYIAQFTSQFLSL (SEQ ID NO: 26) (FIGS. 4B, 4C).

As shown by FIG. 4D, CD4⁺ T-cells released IFN-γ and TNF-α in response to 15-mer peptides 50 EKVKRKKNVLRLCCK (SEQ. ID NO: 21) and 71 EEQYIAQFTSQFLSL (SEQ ID NO: 26) indicating that these peptides were HLA class II-restricted epitopes.

As shown by FIG. 4E, CD8⁺ T-cells shol.ved no specificity to peptides 50 and 71 (FIG. 4E).

The complete data of CD4⁺ restricted T-cell epitopes identified in PRAME is summarized in Table 2.

TABLE 2
Peptide sequences of CD4-restricted T-cell epitopes identified in PRAME

| | Amino | SEQ | | | | | | | | |
|---|---|---|---|---|---|---|---|---|---|---|
| Peptide | acid | ID | HLA- | HLA- | HLA- | HLA- | HLA- | HLA- | HLA- | HLA- |
| sequence | location | NO: | DRB1 | DRB3 | DRB4 | DRB5 | DQA1 | DQB1 | DPA1 | DPB1 |
| RLWGSIQSRYISMSV | 5-19 | 12 | 01:01; | 02:02 | | | 01:01; | 05:01; | 01:03; | 04:01; |
| | | | 11:01 | | | | 05:01 | 05:01 | 01:03 | 04:02 |
| TSPRRLVELAGQSLL | 21-35 | 13 | 01:01; | | | 01:01 | 01:01; | 05:01; | 01:03; | 04:01; |
| | | | 15:01 | | | | 01:02 | 06:02 | 01:03 | 04:01 |
| RLVELAGQSLLKDEA | 25-39 | 14 | 01:01; | | | 01:01 | 01:01; | 05:01; | 01:03; | 04:01; |
| | | | 15:01 | | | | 01:02 | 06:02 | 01:03 | 04:01 |
| PFTCLPLGVLMKGQH | 77-91 | 15 | 07:01; | 02:02 | 01:01 | | 02:01; | 02:01; | 01:03; | 01:01; |
| | | | 11:03 | | | | 05:01 | 03:01 | 02:02 | 04:02 |
| LPLGVLMKGQHLHLE | 81-95 | 16 | 07:01; | 02:02 | 01:01 | | 02:01; | 02:01; | 01:03; | 01:01; |
| | | | 11:03 | | | | 05:01 | 03:01 | 02:02 | 04:02 |
| | | | 01:01; | 02:02 | | | 01:01; | 05:01; | 01:03; | 04:01; |
| | | | 11:01 | | | | 05:01 | 05:01 | 01:03 | 04:02 |
| | | | 08:01; | | | | 04:01; | 03:01; | 01:03; | 04:01; |
| | | | 08:10 | | | | 06:01 | 04:02 | 01:03 | 04:01 |
| | | | 01:85; | 01:01 | | | 01:03; | 05:01; | 01:03; | 04:01; |
| | | | 14:07 | | | | 01:02 | 01:02 | 01:02 | 01:01 |
| VLMKGQHLHLETFKA | 85-99 | 17 | 08:01; | | | | 04:01; | 03:01; | 01:03; | 04:01; |
| | | | 08:10 | | | | 06:01 | 04:02 | 01:03 | 04:01 |
| DVLLAQEVRPRRWKL | 105-119 | 18 | 01:85; | 01:01 | | | 01:03; | 05:01; | 01:03; | 04:01; |
| | | | 14:07 | | | | 01:02 | 01:02 | 01:02 | 01:01 |
| DELFSYLIEKVKRKK | 188-202 | 19 | 07:01; | 02:02 | 01:01 | | 02:01; | 02:01; | 01:03; | 1:01; |
| | | | 11:03 | | | | 05:01 | 03:01 | 02:02 | 04:02 |
| SYLIEKVKRKKNVLR | 193-207 | 20 | 07:01; | 02:02 | 01:01 | | 02:01; | 02:01; | 01:03; | 01:02; |
| | | | 11:03 | | | | 05:01 | 03:01 | 02:02 | 04:02 |
| | | | 08:01; | | | | 04:01; | 03:01; | 01:03; | 04:01; |
| | | | 08:10 | | | | 06:01 | 04:02 | 01:03 | 04:01 |
| EKVKRKKNVLRLCCK | 197-211 | 21 | 08:01; | | | | 04:01; | 03:01; | 01:03; | 04:01; |
| | | | 08:10 | | | | 06:01 | 04:02 | 01:03 | 04:01 |
| | | | 04:02; | | 01:02 | | 01:03; | 03:02; | 01:03; | 04:01; |
| | | | 15:02 | | | | 03:01 | 06:01 | 02:01 | 17:01 |
| CCKKLKIFAMPMQDI | 209-223 | 22 | 01:85; | | | | 01:03; | 05:01; | 01:03; | 04:01; |
| | | | 14:07 | | | | 01:02 | 01:02 | 01:02 | 01:01 |
| AMPMQDIKMILKMVQ | 217-231 | 23 | 01:85; | 01:01 | | | 01:03; | 05:01; | 01:03; | 04:01; |
| | | | 14:07 | | | | 01:02 | 01:02 | 01:02 | 01:01 |
| QDIKMILKMVQLDSI | 221-235 | 24 | 01:85; | 01:01 | | | 01:03; | 05:01; | 01:03; | 04:01; |
| | | | 14:07 | | | | 01:02 | 01:02 | 01:02 | 01:01 |
| SPLGQMINLRRLLL | 253-267 | 25 | 01:85; | 01:01 | | | 01:03; | 05:01; | 01:03; | 04:01; |
| | | | 14:07 | | | | 01:02 | 01:02 | 01:02 | 01:01 |
| | | | 01:01; | | | 01:01 | 01:01; | 05:01; | 01:03; | 04:01; |
| | | | 15:01 | | | | 01:02 | 06:02 | 01:03 | 04:01 |
| EEQYIAQFTSQFLSL | 281-295 | 26 | 04:02; | | 01:02 | | 01:03; | 03:02; | 01:03; | 04:01; |
| | | | 15:02 | | | | 03:01 | 06:01 | 02:01 | 17:01 |

Bold: strong binder (<2)
Underlined: week binder (2-10)

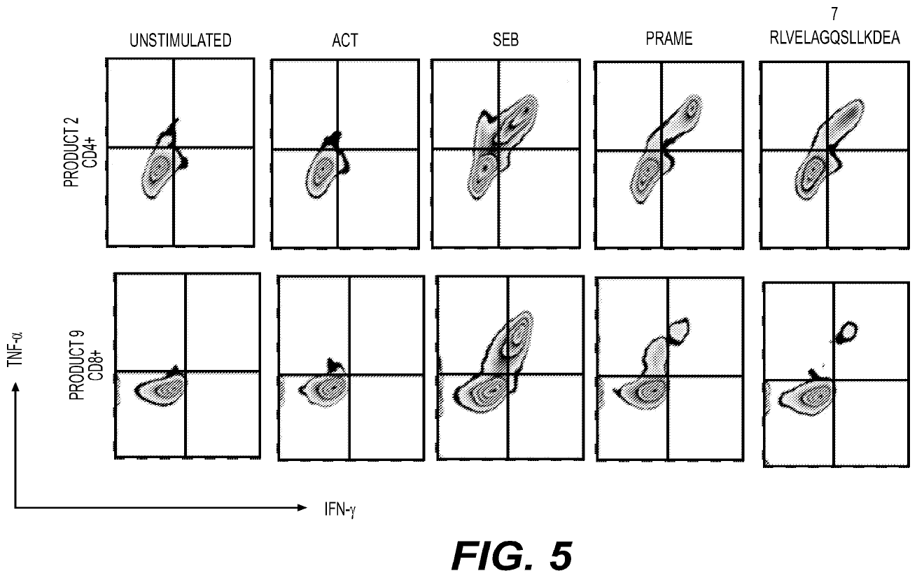

T-cell epitopes elicit both CD4+and CD8+ T-cell responses, As shown by FIG. 5, some PRAME peptides are able to simultaneously induce MHC class I-restricted CD8⁺ and WIC class ll-restricted CD4⁺ T-cell responses. Peptide 7 (RLVELAGQSLLKDEA, SEQ. 11) NO: 14) activated both CD4⁺ and CD8⁺ T-cells in vitro.

Terminology. Terminology used herein is for the purpose of describing particular embodiments only and is not intended to be limiting of the invention.

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references, and does not constitute an admission as to the accuracy of the content of such references.

1. A method for eliciting an immune response in a subject having cancer expressing Melanoma antigen preferentially expressed in tumors (“PRAME”) comprising administering T cells which recognize a PRAME epitope of a peptide having an amino acid sequence consisting of SEQ. ID NO: 2, 1, 3, 4, 5, 6, 7, 8, 9, 10, or 11 or SEQ ID NO: 12, 13, 14, 15, 16, 17, 18, 19, 20, 21, 22, 23, 24, 25, or 26.

2. The method of claim 1. wherein the T cells are autologous to the patient.

3. The method of claim 1, wherein the cells are obtained from a healthy donor who shares at least one HLA class 1 or HLA class 2 antigen or allele with the subject.

4. The method of claim 1, wherein the T cells are obtained from peripheral blood mononuclear cells or from tumor infiltrating lymphocytes.

5. The method of claim 1, wherein the T cells are primed and expanded, or expanded, ex vivo or in vitro.

6. The method of claim 1, wherein the T cells are produced by contacting naive T cells or naive T cell precursors, or by contacting T cells that recognize PRAME , with antigen presenting cells exogenously pulsed with at least one peptide epitope of SEQ ID NOS: 1-26.

7. The method of claim 1, wherein said T cells recognize a peptide epitope of PRAME in a peptide consisting of SEQ ID NOS: 1-11.

8. The method of claim 1, wherein said T cells recognize a peptide epitope of SEQ ID NO: 2, 6, 7, 8 or 9.

9. The method of claim 1, wherein said T cells recognize a peptide epitope of SEQ ID NO: 2 and said subject expresses HLA-A 24:02.

10. The method of claim 1, wherein said T cells recognize a peptide epitope of SEQ ID NO: 6 and said subject expresses HLA-B 35:03.

11. The method of claim 1, wherein said T cells recognize a peptide epitope of SEQ ID NO: 9 and said subject expresses HLA-B 08:02.

12. The method of claim 1. wherein said T cells recognize a peptide epitope of PRAME in a peptide consisting of SEQ ID NOS: 12to 26.

13. The method of claim 1, wherein said T cells recognize a peptide epitope of PRAME in a peptide consisting of SEQ ID NO: 12, 13, 19, 20, 22, 26, 27 or 28.

14. The method of claim 1, wherein said T cells recognize a peptide epitope of PRAME in a peptide consisting of SEQ ID NO: 14.

15. The method of claim 1, wherein said T cells recognize a peptide epitope of SEQ ID NO: 13 and said subject expresses HLA-DRB1 01:01.

16. The method of claim 1, wherein said T cells recognize a peptide epitope of SEQ ID NO: 19 and said subject expresses HLA-DPA1 02:02.

17. The method of claim 1, wherein said T cells recognize a peptide epitope of SEQ ID NO: 19 and said subject expresses HLA-DPB1 04:02.

18. The method of claim 1, wherein said T cells recognize a peptide epitope of SEQ ID NO: 20 and said subject expresses HLA-DRB1 11:03.

19. The method of claim 1, wherein said T cells recognize a peptide epitope of SEQ ID NO: 26 and said subject expresses HiLA-DRB1 15:02.

20. The method of claim 1, wherein said T cells comprise different T cell populations which each recognize a different peptide epitope of PRAME.

21. The method of claim 1, wherein said T cells are obtained from a T cell bank and are selected to comprise at least one HLA class 1 or HLA class 2 molecule shared by a donor and by the subject.

22. The method of claim 1, wherein said T cells are administered in a form of a composition.

23. The method of claim 22, wherein the composition further comprises an adjuvant.

24. The method of claim 23, wherein the adjuvant is selected from anti-CD40 antibody, imiquimod, resiquimod, GM-CSF, cyclophosphamide, sunitinib, bevacizumab, interferon-alpha, interferon-beta, CpG oligonucleotides and derivatives, poly-(I:C) and derivatives, RNA, sildenafil, particulate formulations with poly(lactide co-glycolide) (PLG), virosomes, interleukin (IL)-1, IL-2, IL-4, IL-7, IL-12, IL-13, IL-15, IL-21, and IL-23.

25. The method of claim 1, wherein the cancer is a hematopoietic neoplasia.

26. The method of claim 1. wherein the cancer is a leukemia.

27. The method of claim I, wherein the cancer is a solid cancer.

28. The method of claim 1, wherein the cancer is selected from the group consisting of melanoma, lymphoma, papillomas, breast or cervical carcinomas, acute and chronic leukemia, medulloblas , non-small cell lung carcinoma, head and neck cancer, renal carcinoma, pancreatic carcinoma, prostate cancer, small cell lung cancer, multiple myeloma, sarcomas and hematological malignancies like chronic myeloid leukemia and acute myeloid leukemia.

29. The method of claim 1, wherein the patient has been treated for cancer and has minimal residual disease.

30. The method of claim 1, wherein said T cells further comprise a population of T cells recognizing at least one antigen selected from the group consisting of NYESO, MAGE A4, MAGE A3, MAGE A1, Survivin, WT1, neuroelasta.se, proteinase 3, p53, CEA, claudin6, Histone H1, Histone H2 Histone H3, Histone H4, MART1, gp100, SOX2, SSX2, Nanog, Oct4, Myc, and Ras.

31. A method for producing T cells which recognize PRAME comprising:
contacting a T cell or precursor T cell pith an antigen presenting cell that presents at least one peptide epitope of 1?It.ANTIH, and
recovering a population of T cells recognizing PRAME; wherein said peptide epitope of PRAME is present in a peptide having an amino acid sequence consisting of one or more of SEQ ID NOS: 1-26.

32. The method of claim 31, wherein said T cell or precursor T cell and said antigen presenting cell are autologous.

33. The method of claim 31, wherein said T cell or precursor T cell and said antigen presenting cell share at least one HLA class 1 or FHA class 2 antigen.

34. The method of claim 31, wherein said. T cells or precursor T cells are from a sub ect to PRAME.

35. The method of claim 31, wherein said T cells or precursor T cells are memory T cells or effector T cells which recognize PRAME.

36. The method of claim 31, further comprising separating the T cells which recognize by PRAME into suhpopulations of T cells expressing one or more markers distinctive for that suhpopulation.

37. The method of claim 31, further comprising suspending the T cells which recognize PRAME in a storage buffer or in a cryogenic medium, and storing or freezing viable T cells for later use.

38. A composition comprising an isolated population of T cells that recognize a peptide epitope of MAW, described by any one of the peptides of SEQ :ID NOS: 1-26when presented by a matched HLA class 1 or HLA class II protein in combination with an artificial medium or carrier that maintains viability of the T cells.

39. The composition according to claim 38 further comprising an adjuvant or cytokine,

40. Use of a population of T cells that recognize a peptide epitope of PRAME described by any one of the peptides of SEQ ID NOS; 1-26, when said peptide epitope is presented by an EILA class I or I-ILA class II protein, for preparation of a medicament to treat a neoplasm or cancer expressing 1?ItAME,

41. The use according to claim 40, wherein the cancer expresses PRAME or is selected from the group consisting of melanoma, lymphoma, papillomas, breast or cervical carcinomas, acute and chronic leukemia, medulloblastoma, non-small cell lung carcinoma, head and neck cancer, renal carcinoma, pancreatic carcinoma, prostate cancer, small cell lung cancer, multiple myeloma, sarcomas and hematological malignancies like chronic myeloid leukemia and acute myeloid leukemia.

42. A peptide or covalently modified peptide comprising an amino acid sequence of any one of SEQ :ID -NOS: 1-26.

43. The peptide or modified peptide of claim 42 that has been covalently modified to increase its biological half-life in vivo when administered to a subject.

44. Use of a peptide or covalently modified peptide according to claim 42 for the manufacture of a medicament, preferably a vaccine for the treatment or prevention of cancer.

45. A use according to claim 44, wherein the cancer expresses PRAME or is selected from the group consisting of melanoma, lymphoma, papillomas, breast or cervical carcinomas, acute and chronic leukemia, medulloblastoma, non-small cell lung carcinoma, head and neck cancer, renal carcinoma, pancreatic carcinoma, prostate cancer, small cell lung cancer, multiple myeloma, sarcomas and hematological malignancies like chronic myeloid leukemia and acute myeloid leukemia.

46. A composition comprising the peptide or covalently-modified peptide of claim 42 and a pharmaceutically acceptable adjuvant, carrier, excipient, and/or adjuvant.

47. The composition of claim 46, wherein said peptide or covalently-modified peptide has a length of no more than 25 contiguous amino acid residues.

48. The composition of claim 46, wherein said peptide or covalently-modified peptide has a length of no more than 15 contiguous amino acid residues.

49. The composition of claim 46 that comprises two or more peptides each comprise a different amino acid sequence according to SEQ NOS: 1-26.

50. A method for treating a subject having cancer expressing PRAME comprising administering the composition of claim 43 to said subject, optionally, in combination with an adjuvant or immunological carrier,

51. The method of claim 50, wherein said composition is administered in combination with antigen presenting cells which restrict said peptide by an MLA class 1 or ELLA class 2 antigen shared with the subject.

52. An artificial polynucleotide construct that encodes at least one peptide comprising an amino acid sequence of SEQ. II) NOS: 1-26, wherein said amino acid sequence is no longer than 50 contiguous amino acid residues.

53. A vector or host cell comprising the artificial polynucleotide construct of claim 52.

54. A cell comprising the artificial polynucleotide construct of claim 52 that expresses at least one HLA class 1 or HLA class 2 antigen which restricts the peptide encoded by said artificial polynucleotide construct.

55. Use of an artificial polynucleotide construct according to claim 52 for the manufacture of a medicament, preferably for manufacture of a nucleic acid based vaccine for the treatment or prevention of cancer.

56. A use according to claim 55. wherein the cancer expresses PRAMS or is selected from the group consisting of melanoma, lymphoma, papillomas, breast or cervical carcinomas, acute and chronic leukemia, medulloblastoma, non-small cell lung carcinoma, head and neck cancer, renal carcinoma, pancreatic carcinoma, prostate cancer, small cell lung cancer, multiple myeloma, sarcomas and hematological malignancies like chronic myeloid leukemia and acute myeloid leukemia.
